(19) United States
(12) Patent Application Publication
(10) Pub. No.: US 2022/0409606 A1
(43) Pub. Date: Dec. 29, 2022

(54) TREATMENT OF NEURODEGENERATION VIA REPROGRAMMING METABOLISM BY INHIBITING PHD

(71) Applicant: The Trustees of Columbia University in the City of New York, New York, NY (US)

(72) Inventors: Stephen H. Tsang, New York, NY (US); Xuan Cui, New York, NY (US); Christine Xu, New York, NY (US); Karen Sophia Park, New York, NY (US)

(21) Appl. No.: 16/622,029

(22) PCT Filed: Jun. 15, 2018
(86) PCT No.: PCT/US2018/037731

§ 371 Date: Dec. 12, 2019

Related U.S. Application Data
(60) Provisional application No. 62/520,261, filed on Jun. 15, 2017.
(60) Provisional application No. 62/545,260, filed on Aug. 14, 2017.

(51) Int. Cl. A61K 31/472; A61K 31/50; A61K 31/5377; A61K 9/00; A61P 25/28; A61P 27/06

(52) U.S. Cl. CPC A61K 31/472; A61K 31/50; A61K 31/5377; A61K 9/0019; A61P 25/28; A61P 27/06

(57) ABSTRACT

The present disclosure relates to methods and compounds for promoting anabolic pathways in neuronal cells leading to improved neuronal survival. In particular, the present disclosure relates to inhibiting PHD to promote glycolysis and neuronal survival in a variety of neurodegenerative conditions such as retinitis pigmentosa.

### CROSS-REFERENCE TO RELATED APPLICATION

The present application is a U.S. National Phase Application under 35 U.S.C. § 371 of International Patent Application No. PCT/US2018/037731, filed Jun. 15, 2018, which claims the priority to U.S. Provisional Patent Application Ser. Nos. 62/520,261, filed on Jun. 15, 2017 and 62/545,260, filed on Aug. 14, 2017, each of which is incorporated by reference as if expressly set forth in its entirety herein.

### FIELD OF THE INVENTION

The present disclosure relates to methods and compounds for promoting anabolic pathways in neuronal cells leading to improved neuronal survival. In particular, the present disclosure relates to inhibiting PHD/Phd to promote glycolysis and neuronal survival in a variety of neurodegenerative conditions, and specifically in retinitis pigmentosa.

### BACKGROUND

Retinitis pigmentosa (RP) is a hereditary disorder causing retinal degeneration. RP leads to progressive photoreceptor dysfunction, dysmorphosis and symptoms such as nyctalopia, tunnel vision and eventually, blindness. This disease is estimated to affect nearly 1 million people worldwide and leads to a substantial decrease in the ability of affected individuals to lead independent lives and conduct activities of daily living.

A heterogeneous genetic condition, RP is linked to more than 60 genes, most of which are exclusively expressed in rod photoreceptors. Daiger, et al., (2008) Mutations in known genes account for 58% of autosomal dominant retinitis pigmentosa (adRP). Adv. Exp. Med. Biol., 613, 203-209. RP is an incurable neurodegenerative condition. Due to the genetic diversity of RP, any therapy that is gene specific can only benefit a small fraction of patients with RP. There is currently no effective therapeutic option for patients with RP or any other patient with a retinal degenerative disease, including atrophic age-related macular degeneration (AMD), which affects more than 1.5 million individuals in the United States. While some disease management options are available for patients with wet AMD, this group represents only about 10% of all AMD cases, and there is no effective treatment for the dry (atrophic) form. Photoreceptor loss is one of the earliest pathological developments of AMD.

Thus, there is an urgent need for additional therapeutics as well as more broadly effective gene therapies for alleviating retinal degenerative diseases such as RP and AMD, and more broadly for promoting neuronal survival in neurodegenerative diseases such as glaucoma, Alzheimer' s, Parkinson's, Huntington's, Amyotrophic lateral sclerosis (ALS), Lewy body dementia, and similar neurodegenerative conditions or other conditions that would benefit from upregulating anabolism and downregulating catabolism to promote neuronal survival.

### SUMMARY

The present disclosure provides for a method of increasing survival of a neuronal cell. The method may comprise inhibiting or decreasing level and/or activity of PHD in the neuronal cell.

The present disclosure provides for a method of increasing neuronal survival in a patient in need thereof. The method may comprise altering glycolysis by inhibiting or decreasing level and/or activity of PHD in a neuronal cell.

Also encompassed by the present disclosure is a method of increasing photoreceptor survival. The method may comprise altering glycolysis by level and/or activity of PHD in a photoreceptor cell.

The inhibiting or decreasing may comprise administering an effective amount of an inhibitor of PHD. The inhibiting or decreasing may comprise administering an effective amount of any combination of inhibitors of PHD.

The present disclosure provides for a method for treating a neurodegenerative condition in a subject. The method may comprise administering a therapeutically effective amount of an inhibitor of prolyl hydroxylase (PHD) to the subject.

The neuronal cell may be a cone cell or a rod cell, or a combination of cone cells, rod cells, and/or other retinal cells.

The photoreceptor cell may be a cone cell, a rod cell, or a retinal cell, or a combination of cone cells, rod cells, and/or retinal cells.

The inhibitors of PHD may be proteins, nucleic acids, chemicals or combinations thereof. Non-limiting examples of the inhibitors of PHD include Roxadustat (FG-4592), Vadadustat (AKB-6548), Daprodustat (GSK1278863), Desidustat (ZYAN-1), Molidustat (Bay 85-3934), MK-8617, YC-1, IOX-2, 2-methoxyestradiol, GN-44028, AKB-4924, Bay 87-2243, FG-2216, FG-4497, or combinations thereof. Non-limiting examples of the inhibitors of PHD also include the compounds in Table 1.

The nucleic acid inhibitors of PHD may be an antisense oligonucleotide, a small interfering RNA (siRNA), a short hairpin RNA (shRNA), a guide RNA (gRNA), or combinations thereof.

The patient may suffer from at least one retinal degenerative disease. The retinal degenerative diseases include, but are not limited to, retinitis pigmentosa (RP), age-related macular degeneration (AMD), and/or glaucoma.

The patient may suffer from at least one neurodegenerative disease. The neurodegenerative diseases include, but are not limited to, Alzheimer's disease, Parkinson's disease, Huntington's disease, Amyotrophic lateral sclerosis (ALS), and/or Lewy body dementia.

The inhibitor of PHD may be administered by intravitreal injection, or by subretinal injection.

### BRIEF DESCRIPTION OF THE DRAWINGS

FIG. 1A includes histology images showing a deceleration in photoreceptor degeneration in the treated group where an inducible, floxed-CreER, Pde6b mouse model of RP was used to genetically enhance glycolysis in photoreceptors.

FIG. 1B shows that electroretinogram outcomes are always higher in the treated (white bar) vs. untreated group (black bar). In the treated group, an inducible, floxed-CreER, Pde6b mouse model of RP was used to genetically enhance glycolysis in photoreceptors.

FIG. 1C shows that high-energy molecules like ATP and GTP were found in greater abundance in the treated group compared to the untreated group, suggesting upregulated glycolysis. In the treated group, an inducible, floxed-CreER, Pde6b mouse model of RP was used to genetically enhance glycolysis in photoreceptors.

FIG. 2 shows glycolysis flux in 20 mg/kg FG-4592 vs. control. A PHD inhibitor/antagonist, FG-4592, was administered to a RP mouse.

### DETAILED DESCRIPTION

The present disclosure provides for a non-gene-specific strategy for treating all RP patients, regardless of their genetic background. We have found that mutant rod photoreceptors have a high energy demand resulting in dysregulated glucose metabolism in RP retinas. Thus, restoring the metabolic balance can prevent photoreceptor death and vision loss. The present disclosure provides for compositions and methods for inhibiting PHD in the treatment or prophylaxis of retinal degenerative diseases, such as retinitis pigmentosa (RP), age-related macular degeneration (AMD), and glaucoma, or neurodegenerative diseases.

Methods and compositions of the present invention can be used for prophylaxis as well as treating a retinal degenerative disease or a neurodegenerative disease (e.g., amelioration of signs and/or symptoms of the retinal degenerative disease or neurodegenerative disease).

For prophylaxis, the present composition can be administered to a subject in order to prevent the onset of one or more symptoms of retinal degenerative disease. In one embodiment, the subject can be asymptomatic. A prophylactically effective amount of the agent or composition is administered to such a subject. A prophylactically effective amount is an amount which prevents the onset of one or more symptoms of the retinal degenerative disease.

The present compositions may be used in vitro or administered to a subject. The administration may be topical, intravenous, intranasal, or any other suitable route as described herein. The present compositions may be administered by intravitreal injection or subretinal injection.

The subject/patient treated with the present method and composition may suffer from one or more retinal degenerative diseases such as retinitis pigmentosa (RP), age-related macular degeneration (AMD), or glaucoma, or one or more neurodegenerative diseases including Alzheimer's, Parkinson's, Huntington's, Amyotrophic lateral sclerosis (ALS), or Lewy body dementia.

We have discovered that promoting glycolysis can slow and even transiently halt cells from dying in neurodegenerative mouse models. Specifically, we demonstrated that knocking out Sirtuin6, an inhibitor of glycolysis, can achieve a rescue effect in mouse models of retinitis pigmentosa, a blinding condition. Prolyl hydroxylase (PHD) can regulate metabolism. By knocking out Phd, we found that we can slow retinal degeneration. Currently, diseases of the central nervous system, like retinitis pigmentosa, age-related macular degeneration (AMD), Alzheimer's, and Parkinson's, are not treatable. The strategy of downregulating catabolism and upregulating anabolism can be used to treat, or treat prophylactically, these conditions.

Non-limiting examples of PHDs include, PHD1/EGLN2/HPH3, PHD2/EGLN1/HPH2, PHD3/EGLN3/HPH1 and PHD4/P4H-TM/EGLN4.

A small molecule drug may be used to increase glycolysis in a neuronal cell, such as a rod. An inhibitor or antagonist of a prolyl hydroxylase (PHD) may be used to treat a neurodegenerative condition.

In one embodiment, a PHD inhibitor may be tested in an RP mouse model to assess outcomes.

Non-limiting examples of inhibitors of PHD are in Table 1 (Hypoxia-Inducible Factor-Prolyl Hydroxylase (HIF-PH) Inhibitors).

The amount and/or activity of PHD may be downregulated by RNA interference or RNAi (such as small interfering RNA or siRNAs, a small hairpin RNA or shRNAs, microRNA or miRNAs, a double-stranded RNA (dsRNA), etc.), antisense molecules, and/or ribozymes targeting the DNA or mRNA encoding PHD. The amount and/or activity of PHD may be downregulated by gene knockout. The amount and/or activity of PHD may be downregulated by cluster regularly interspaced short palindromic repeat-associated nuclease (CRISPR) technology.

In certain embodiments, the length of the rescue effect is monitored. In certain embodiments, small molecules that are able to induce inhibition of PHD in a similar fashion as is achieved via gene therapy are used to treat a neurodegenerative condition.

The present method and composition may be used to treat other neurodegenerative condition, such as AMD, glaucoma, Alzheimer's disease and Parkinson's disease.

In accordance with the present disclosure, there may be numerous tools and techniques within the skill of the art, such as those commonly used in molecular immunology, cellular immunology, pharmacology, and microbiology. See, e.g., Sambrook et al. (2001) Molecular Cloning: A Laboratory Manual. 3rd ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.

While not wishing to be bound by theory, aspects of the present disclosure relate to methods for increasing anabolism and decreasing catabolism in desired cells, e.g., in neuronal cells. Embodiments of the present disclosure relate to increasing glycolysis in neuronal cells, leading to improved neuronal cell survival. Additional embodiments of the present disclosure relate to methods of increasing photoreceptor cell survival in desired patient populations, including in patients with retinal degenerative diseases described herein, such as RP, AMD, and glaucoma. This strategy may also be used in combination with gene therapies and neurotrophic factor administration for heightened treatment efficacy.

By “neuronal” is meant to refer to and include any cells which compose the central or peripheral nervous system. (See, Dowling J E. The retina: an approachable part of the brain. Rev. ed. Cambridge, Mass.: Belknap Press of Harvard University Press; 2012.)

By “retinal” is meant to refer to and include any light-sensitive cells in the eye as well as the supporting cells that enable, facilitate, or are related to the phototransduction cascade.

By “nucleic acid” or “nucleic acid molecule” is meant to include a DNA, RNA, mRNA, cDNA, or recombinant DNA or RNA.

By “animal” is meant any member of the animal kingdom including vertebrates (e.g., frogs, salamanders, chickens, or horses) and invertebrates (e.g., worms, etc.). Preferred animals are mammals. Preferred mammalian animals include livestock animals (e.g., ungulates, such as bovines, buffalo, equines, ovines, porcines and caprines), as well as rodents (e.g., mice, hamsters, rats and guinea pigs), canines, felines and primates. By “non-human” is meant to include all animals, especially mammals and including primates other than human primates.

By “medium” or “media” is meant the nutrient solution in which cells and tissues are grown.

The term “pharmaceutically acceptable carrier”, as used herein means a pharmaceutically-acceptable material, composition or vehicle, such as a liquid or solid filler, diluent, excipient, solvent or encapsulating material, involved in carrying or transporting a chemical agent. The diluent or carrier ingredients should not be such as to diminish the therapeutic effects of the active compound(s).

The term “composition” as used herein means a product which results from the mixing or combining of more than one element or ingredient.

“Treating” or “treatment” of a state, disorder or condition includes:

(1) preventing or delaying the appearance of clinical symptoms of the state, disorder, or condition developing in a person who may be afflicted with or predisposed to the state, disorder or condition but does not yet experience or display clinical symptoms of the state, disorder or condition; or

(2) inhibiting the state, disorder or condition, i.e., arresting, reducing or delaying the development of the disease or a relapse thereof (in case of maintenance treatment) or at least one clinical symptom, sign, or test, thereof; or

(3) relieving the disease, i.e., causing regression of the state, disorder or condition or at least one of its clinical or sub-clinical symptoms or signs.

The benefit to a subject to be treated is either statistically significant or at least perceptible to the patient or to the physician.

“Treat” or “treating” may refer to administering a therapeutic agent, such as a composition containing any of the tissue-specific, e.g., neuronal or ocular targeted viral vectors, RNAi, shRNA or other PHD inhibitors, combinations thereof, or similar compositions described herein, internally or externally to a subject or patient having one or more disease symptoms, or being suspected of having a disease or being at elevated at risk of acquiring a disease, for which the agent has therapeutic activity. Gene editing technology such as CRISPR/Cas9 methods may also be utilized to carry out tissue-specific reduction of PHD or a combination thereof. Typically, the agent is administered in an amount effective to alleviate one or more disease symptoms in the treated subject or population, whether by inducing the regression of or inhibiting the progression of such symptom(s) by any clinically measurable degree. The amount of a therapeutic agent that is effective to alleviate any particular disease symptom (also referred to as the “therapeutically effective amount”) may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom. While an embodiment of the present invention (e.g., a treatment method or article of manufacture) may not be effective in alleviating the target disease symptom(s) in every subject, it should alleviate the target disease symptom(s) in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“Treatment,” as it applies to a human, veterinary, or research subject, refers to therapeutic treatment, prophylactic or preventative measures, to research and diagnostic applications. “Treatment” as it applies to a human, veterinary, or research subject, or cell, tissue, or organ, encompasses transfection of any of the tissue-targeted viral vectors, delivery of RNAi, shRNA or other PHD inhibitors, combinations thereof, or similar compositions, including gene editing technology such as CRISPR/cas9 methods, which may be utilized to carry out tissue specific reduction of PHD, combinations thereof or related methods described herein as applied to a human or animal subject, a cell, tissue, physiological compartment, or physiological fluid.

A “therapeutically effective amount” means the amount of a compound that, when administered to an animal for treating a state, disorder or condition, is sufficient to effect such treatment. The “therapeutically effective amount” will vary depending on the compound, the disease and its severity and the age, weight, physical condition and responsiveness of the animal to be treated.

“Patient” or “subject” refers to mammals and includes human and veterinary subjects.

Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopoeia or other generally recognized pharmacopeias for use in animals, and more particularly in humans.

In certain embodiments, the methods of the present disclosure can be used for arresting progression of, or ameliorating, vision loss associated with photoreceptor degeneration including retinitis pigmentosa (RP) and age-related macular degeneration (AMD) in the subject. Vision loss linked to retinitis pigmentosa may include decrease in peripheral vision, central (reading) vision, night vision, day vision, loss of color perception, loss of contrast sensitivity, or reduction in visual acuity. The methods of the present disclosure can also be used to prevent, or arrest photoreceptor function loss, or increase photoreceptor function in the subject.

RP is diagnosed in part, through an examination of the retina and genetic testing. The eye exam usually reveals abnormal, intraretinal pigment migration. Additional tests for diagnosing RP include electroretinogram (ERG) and visual field testing.

Methods for measuring or assessing visual function, retinal function (such as responsiveness to light stimulation), or retinal structure in a subject are well known to one of skill in the art. See, e.g. Kanski's Clinical Ophthalmology: A Systematic Approach, Edition 8, Elsevier Health Sciences, 2015. Methods for measuring or assessing retinal response to light include may include detecting an electrical response of the retina to a light stimulus. This response can be detected by measuring an electroretinogram (ERG; for example full-field ERG, multifocal ERG, or ERG photostress test), visual evoked potential, or optokinetic nystagmus (see, e.g., Wester et al., Invest. Ophthalmol. Vis. Sci. 48:4542-4548, 2007). Furthermore, retinal response to light may be measured by directly detecting retinal response (for example by use of a microelectrode at the retinal surface). ERG has been extensively described by Vincent et al. Retina, 2013 Jan; 33(1):5-12. Thus, methods of the present disclosure can be used to improve visual function, retinal function (such as responsiveness to light stimulation), retinal structure, or any other clinical symptoms or phenotypic changes associated with ocular diseases in subjects afflicted with ocular disease.

The dosage of the therapeutic formulation will vary widely, depending upon the nature of the disease, the patient's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level. In some cases, oral administration will require a higher dose than if administered intravenously. In some cases, topical administration will include application several times a day, as needed, for a number of days or weeks in order to provide an effective topical dose.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, olive oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

As used herein, the term “adjuvant” refers to a compound or mixture that enhances the immune response to an antigen. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, Calif., p. 384). Often, a primary challenge with an antigen alone, in the absence of an adjuvant, will fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, and potentially useful human adjuvants such as N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine, and BCG (bacille Calmette-Guerin). Preferably, the adjuvant is pharmaceutically acceptable.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g.: enhancers, kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Moreover, inducible and tissue specific expression of an RNA, transmembrane proteins, or other proteins can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various commercially available ubiquitous as well as tissue-specific promoters can be found at http://www.invivogen.com/prom-a-list and https://www.addgene.org/. In addition, promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

Vectors according to the present disclosure can be transformed, transfected or otherwise introduced into a wide variety of host cells. Transfection refers to the taking up of a vector by a host cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral transduction, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

In certain embodiments, the methods described herein can be utilized to treat ocular disease, neuronal disease, or improve photoreceptor function in a patient and can comprise administering to the patient an effective concentration of a composition comprising any of the recombinant AAVs described herein and a pharmaceutically acceptable carrier. In one embodiment, an effective concentration of virus is 1×10⁶-11×10¹³ GC/ml. The range of viral concentration effective for the treatment can vary depending on factors including, but not limited to specific mutation, patient's age, and other clinical parameters.

Production of recombinant AAV vectors and their use in in vitro and in vivo administration has been discussed in detail by Gray et al. (Curr Protoc Neurosci. 2011 Oct, Chapter:Unit 4.17).

The recombinant AAV containing the desired recombinant DNA can be formulated into a pharmaceutical composition intended for subretinal or intravitreal injection. Such formulation involves the use of a pharmaceutically and/or physiologically acceptable vehicle or carrier, particularly one suitable for administration to the eye, e.g., by subretinal injection, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline.

In one embodiment, the carrier is an isotonic sodium chloride solution. In another embodiment, the carrier is balanced salt solution. In one embodiment, the carrier includes tween. If the virus is to be stored long-term, it may be frozen in the presence of glycerol or Tween-20. In another embodiment, the pharmaceutically acceptable carrier comprises a surfactant, such as perfluorooctane (Perfluoron liquid). In certain embodiments, the pharmaceutical composition described above is administered to the subject by subretinal injection. In other embodiments, the pharmaceutical composition is administered by intravitreal injection. Other forms of administration that may be useful in the methods described herein include, but are not limited to, direct delivery to a desired organ (e.g., the eye), oral, inhalation, intranasal, intratracheal, intravenous, intramuscular, subcutaneous, intradermal, and other parental routes of administration. Additionally, routes of administration may be combined, if desired.

In some embodiments, route of administration is subretinal injection or intravitreal injection.

### Inhibitors of PHD

The hypoxia inducible factor (HIF) is one of the key regulators of oxygen homeostasis. At normal oxygen levels, the alpha subunit of HIF is targeted for degradation by prolyl hydroxylation. The alpha subunits of HIF are hydroxylated at conserved proline residues by HIF prolyl hydroxylases, allowing their recognition and ubiquitination, which labels them for rapid degradation by the proteasome. Maxwell et al., 1999, Nature. 399 (6733): 271-5. In hypoxic conditions, HIF prolyl hydroxylase is inhibited, since it utilizes oxygen as a co-substrate. Semenza, 2004, Physiology, 19 (4): 176-82. In normal circumstances after injury, HIF-1a is degraded by prolyl hydroxylases (PHDs). HIF-1 is a heterodimeric protein that consists of two subunits, HIF-1α and HIF-1β. Whereas HIF-1β is constitutively expressed, the expression of HIF-1a is induced by oxygen concentrations below 6%. HIF-1 heterodimers bind to the hypoxia response element (HRE), a 5-RCGTG-3 consensus sequence.

Hypoxia-inducible factor prolyl hydroxylase (HIF prolyl hydroxylase) is also known as prolyl hydroxylase domain-containing protein or prolyl hydroxylase domain protein which may be abbreviated as PHD. There are at least three different PHD isoforms, PHD1, PHD2, and PHD3, also referred to as EGLN2, EGLN1, and EGLN3, or HPH3, HPH2, and HPH1, respectively. Bruick et al., Science 294:1337-40 (2001); Epstein et al., Cell 107:43-54 (2001); Ivan et al., Proc. Natl. Acad. Sci. USA 99:13459-464 (2002); Lieb et al., Biochem. Cell. Biol. 80:421-426 (2002); Huang et al., J. Biol. Chem. 277:39792-800 (2002)). A widely expressed fourth PHD/HPH has also been identified (Oehme et al., Biochem. Biophys. Res. Commun. 296(2):343-49 (2002).

Non-limiting examples of PHDs include, PHD1/EGLN2/HPH3, PHD2/EGLN1/HPH2, PHD3/EGLN3/HPH1 and PHD4/P4H-TM/EGLN4.

By “PHD,” “PHD” “Phd,” “Phd” is meant to include the DNA, RNA, mRNA, cDNA, recombinant DNA or RNA, or the protein arising from the gene. As used herein, PHD can refer to the gene or the protein encoded for by the gene, as appropriate in the specific context utilized. Additionally, in certain contexts, the reference will be to the mouse gene or protein, and in others the human gene or protein as appropriate in the specific context.

Any isoform of any PHD may be inhibited by the present inhibitors.

The present inhibitors may target the wild-type or mutant form of PHD.

As used herein, the term “inhibitor” refers to agents capable of down-regulating or otherwise decreasing or suppressing the amount/level and/or activity of PHD.

The mechanism of inhibition may be at the genetic level (e.g., interference with or inhibit expression, transcription or translation, etc.) or at the protein level (e.g., binding, competition, etc.).

A wide variety of suitable inhibitors may be employed, guided by art-recognized criteria such as efficacy, toxicity, stability, specificity, half-life, etc.

### Small Molecule Inhibitors

As used herein, the term “small molecules” encompasses molecules other than proteins or nucleic acids without strict regard to size. Non-limiting examples of small molecules that may be used according to the methods and compositions of the present invention include, small organic molecules, peptide-like molecules, peptidomimetics, carbohydrates, lipids or other organic (carbon containing) or inorganic molecules.

Non-limiting examples of inhibitors of PHD are in Table 1 (Hypoxia-Inducible Factor-Prolyl Hydroxylase (HIF-PH) Inhibitors).

Non-limiting examples of HIF prolyl hydroxylase inhibitors (or PHD inhibitors) include, Roxadustat (FG-4592); Vadadustat (AKB -6548), Daprodustat (GSK1278863), Desidustat (ZYAN-1), Molidustat (Bay 85-3934), MK-8617, YC-1, IOX-2, 2-methoxyestradiol, GN-44028, AKB-4924, Bay 87-2243, FG-2216 and FG-4497. Bruegge et al., (2007) Current Medicinal Chemistry. 14 (17): 1853-62. Becker et al., (2017) Advances in Therapy, 34 (4): 848-853. Pergola et al., (2016) Kidney International, 90 (5): 1115-1122. Ariazi et al., (2017) The Journal of Pharmacology and Experimental Therapeutics, 363 (3): 336-347. Kansagra et al., (2017) Clinical Pharmacokinetics, 57: 87-102. Maxwell et al., (2016) Nature Reviews Nephrology, 12 (3): 157-68. Flamme et al., (2014) PLOS One. 9 (11): e111838. Debenham et al., (2016) Journal of Medicinal Chemistry, 59 (24): 11039-11049. Yeo et al., (2003) Journal of the National Cancer Institute, 95 (7): 516-25. Deppe et al., (2016) Archives of Toxicology. 90 (5): 1141-50. Wang et al., (2011) Current Medicinal Chemistry, 18 (21): 3168-89. Minegishi et al., (2013) ACS Medicinal Chemistry Letters, 4 (2): 297-301. Gortz et al., (2016) The Journal of Clinical Endocrinology and Metabolism, 101 (12): 4834-4842. Beuck et al., (2012) Drug Test Anal., 4: 830-45. Silva et al., (2015) Expert Review of Respiratory Medicine, 9 (4): 405-9. Okumura et al., (2012) Journal of Molecular Medicine, 90 (9): 1079-89.

### Endonucleases

Methods for modification of genomic DNA are well known in the art. For example, methods may use a DNA digesting agent to modify the DNA by either the non-homologous end joining DNA repair pathway (NHEJ) or the homology directed repair (HDR) pathway. The term “DNA digesting agent” refers to an agent that is capable of cleaving bonds (i.e. phosphodiester bonds) between the nucleotide subunits of nucleic acids.

In one embodiment, the DNA digesting agent is a nuclease. Nucleases are enzymes that hydrolyze nucleic acids. Nucleases may be classified as endonucleases or exonucleases. An endonuclease is any of a group of enzymes that catalyze the hydrolysis of bonds between nucleic acids in the interior of a DNA or RNA molecule. An exonuclease is any of a group of enzymes that catalyze the hydrolysis of single nucleotides from the end of a DNA or RNA chain. Nucleases may also be classified based on whether they specifically digest DNA or RNA. A nuclease that specifically catalyzes the hydrolysis of DNA may be referred to as a deoxyribonuclease or DNase, whereas a nuclease that specifically catalyses the hydrolysis of RNA may be referred to as a ribonuclease or an RNase. Some nucleases are specific to either single-stranded or double-stranded nucleic acid sequences. Some enzymes have both exonuclease and endonuclease properties. In addition, some enzymes are able to digest both DNA and RNA sequences.

PHD may be inhibited by using a sequence-specific endonuclease that target the gene encoding PHD.

Non-limiting examples of the endonucleases include a zinc finger nuclease (ZFN), a ZFN dimer, a ZFNickase, a transcription activator-like effector nuclease (TALEN), or a RNA-guided DNA endonuclease (e.g., CRISPR/Cas9). Meganucleases are endonucleases characterized by their capacity to recognize and cut large DNA sequences (12 base pairs or greater). Any suitable meganuclease may be used in the present methods to create double-strand breaks in the host genome, including endonucleases in the LAGLIDADG and PI-Sce family.

The sequence-specific endonuclease of the methods and compositions described herein can be engineered, chimeric, or isolated from an organism. Endonucleases can be engineered to recognize a specific DNA sequence, by, e.g., mutagenesis. Seligman et al. (2002) Mutations altering the cleavage specificity of a homing endonuclease, Nucleic Acids Research 30: 3870-3879. Combinatorial assembly is a method where protein subunits form different enzymes can be associated or fused. Arnould et al. (2006) Engineering of large numbers of highly specific homing endonucleases that induce recombination to novel DNA targets, Journal of Molecular Biology 355: 443-458. These two approaches, mutagenesis and combinatorial assembly, may be combined to produce an engineered endonuclease with desired DNA recognition sequence.

The sequence-specific nuclease can be introduced into the cell in the form of a protein or in the form of a nucleic acid encoding the sequence-specific nuclease, such as an mRNA or a cDNA. Nucleic acids can be delivered to a bacterial cell by transformation, e.g., heat shock, electroporation, etc. In one embodiment, bacterial cells are incubated in a solution containing divalent cations (e.g., calcium chloride) under cold conditions, before being exposed to a heat pulse (heat shock).

One example of a sequence-specific nuclease system that can be used with the methods and compositions described herein includes the CRISPR system (Wiedenheft, B. et al. Nature 482, 331-338 (2012); Jinek, M. et al. Science 337, 816-821 (2012); Mali, P. et al. Science 339, 823-826 (2013); Cong, L. et al. Science 339, 819-823 (2013)). The CRISPR (Clustered Regularly interspaced Short Palindromic Repeats) system exploits RNA-guided DNA-binding and sequence-specific cleavage of target DNA. The guide RNA/Cas combination confers site specificity to the nuclease. A single guide RNA (sgRNA) contains about 20 nucleotides that are complementary to a target genomic DNA sequence upstream of a genomic PAM (protospacer adjacent motifs) site (NGG) and a constant RNA scaffold region. The Cas (CRISPR-associated) protein binds to the sgRNA and the target DNA to which the sgRNA binds and introduces a double-strand break in a defined location upstream of the PAM site. Cas9 harbors two independent nuclease domains homologous to HNH and RuvC endonucleases, and by mutating either of the two domains, the Cas9 protein can be converted to a nickase that introduces single-strand breaks (Cong, L. et al. Science 339, 819-823 (2013)). It is specifically contemplated that the methods and compositions of the present disclosure can be used with the single- or double-strand-inducing version of Cas9, as well as with other RNA-guided DNA nucleases, such as other bacterial Cas9-like systems. The sequence-specific nuclease of the present methods and compositions described herein can be engineered, chimeric, or isolated from an organism. The nuclease can be introduced into the cell in form of a DNA, mRNA and protein. The applications of the CRISPR/Cas system to inhibiting or downregulating PHD can be easily adapted.

In one embodiment, the methods of the present disclosure comprise using one or more sgRNAs to remove, or suppress a glycolysis regulator, such as PHD. In another embodiment, two or more sgRNA(s) are used to remove, or suppress an autosomal dominant disease-related gene.

In one embodiment, the DNA digesting agent can be a site-specific nuclease. In another embodiment, the site-specific nuclease may be a Cas-family nuclease. In a more specific embodiment, the Cas nuclease may be a Cas9 nuclease.

In one embodiment, Cas protein may be a functional derivative of a naturally occurring Cas protein.

In addition to well characterized CRISPR-Cas system, a new CRISPR enzyme, called Cpf1 (Cas protein 1 of PreFran subtype) has recently been described (Zetsche et al. Cell. pii: S0092-8674(15)01200-3. doi: 10.1016/j.cell.2015.09.038 (2015)). Cpf1 is a single RNA-guided endonuclease that lacks tracrRNA, and utilizes a T-rich protospacer-adjacent motif. The authors demonstrated that Cpf1 mediates strong DNA interference with characteristics distinct from those of Cas9. Thus, in one embodiment of the present invention, CRISPR-Cpf1 system can be used to cleave a desired region within the targeted gene.

In further embodiment, the DNA digesting agent is a transcription activator-like effector nuclease (TALEN). TALENs are composed of a TAL effector domain that binds to a specific nucleotide sequence and an endonuclease domain that catalyzes a double strand break at the target site (PCT Patent Publication No. WO2011072246; Miller et al., Nat. Biotechnol. 29, 143-148 (2011); Cermak et al., Nucleic Acid Res. 39, e82 (2011)). Sequence-specific endonucleases may be modular in nature, and DNA binding specificity is obtained by arranging one or more modules. Bibikova et al., Mol. Cell. Biol. 21, 289-297 (2001). Boch et al., Science 326, 1509-1512 (2009).

ZFNs can be composed of two or more (e.g., 2-8, 3-6, 6-8, or more) sequence-specific DNA binding domains (e.g., zinc finger domains) fused to an effector endonuclease domain (e.g., the Fokl endonuclease). Porteus et al., Nat. Biotechnol. 23, 967-973 (2005). Kim et al. (2007) Hybrid restriction enzymes: Zinc finger fusions to Fok I cleavage domain, Proceedings of the National Academy of Sciences of USA, 93: 1156-1160. U.S. Pat. No. 6,824,978. PCT Publication Nos. WO1995/09233 and WO1994018313.

In one embodiment, the DNA digesting agent is a site-specific nuclease of the group or selected from the group consisting of omega, zinc finger, TALE, and CRISPR/Cas.

The sequence-specific endonuclease of the methods and compositions described here can be engineered, chimeric, or isolated from an organism. Endonucleases can be engineered to recognize a specific DNA sequence, by, e.g., mutagenesis. Seligman et al. (2002) Mutations altering the cleavage specificity of a homing endonuclease, Nucleic Acids Research 30: 3870-3879. Combinatorial assembly is a method where protein subunits form different enzymes can be associated or fused. Arnould et al. (2006) Engineering of large numbers of highly specific homing endonucleases that induce recombination to novel DNA targets, Journal of Molecular Biology 355: 443-458. In certain embodiments, these two approaches, mutagenesis and combinatorial assembly, can be combined to produce an engineered endonuclease with desired DNA recognition sequence.

The sequence-specific nuclease can be introduced into the cell in the form of a protein or in the form of a nucleic acid encoding the sequence-specific nuclease, such as an mRNA or a cDNA. Nucleic acids can be delivered as part of a larger construct, such as a plasmid or viral vector, or directly, e.g., by electroporation, lipid vesicles, viral transporters, microinjection, and biolistics. Similarly, the construct containing the one or more transgenes can be delivered by any method appropriate for introducing nucleic acids into a cell.

Single guide RNA(s) used in the methods of the present disclosure can be designed so that they direct binding of the Cas-sgRNA complexes to pre-determined cleavage sites in a genome. In one embodiment, the cleavage sites may be chosen so as to release a fragment or sequence that contains a region of autosomal dominant disease-related gene. In further embodiment, the cleavage sites may be chosen so as to release a fragment or sequence that contains a region of genes encoding glycolysis regulators, e.g. PHD.

For Cas family enzyme (such as Cas9) to successfully bind to DNA, the target sequence in the genomic DNA should be complementary to the sgRNA sequence and must be immediately followed by the correct protospacer adjacent motif or “PAM” sequence. “Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization and promote formation of a CRISPR complex. A target sequence may comprise any polynucleotide, such as DNA or RNA polynucleotides. The Cas9 protein can tolerate mismatches distal from the PAM, however, mismatches within the 12 base pairs (bps) of sequence next to the PAM sequence can dramatically decrease the targeting efficiency. The PAM sequence is present in the DNA target sequence but not in the sgRNA sequence. Any DNA sequence with the correct target sequence followed by the PAM sequence will be bound by Cas9. The PAM sequence varies by the species of the bacteria from which Cas9 was derived. The most widely used CRISPR system is derived from S. pyogenes and the PAM sequence is NGG located on the immediate 3′ end of the sgRNA recognition sequence. The PAM sequences of CRISPR systems from exemplary bacterial species include: Streptococcus pyogenes (NGG), Neisseria meningitidis (NNNNGATT), Streptococcus thermophilus (NNAGAA) and Treponema denticola (NAAAAC).

sgRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31 , 32, 33, 34, 35, 36, 37, 38, 39, 40, 41, 42, 43, 44, 45, 46, 47, 48, 49, 50, 51 , 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81 , 82, 83, 84, 85, 86, 87, 88, 89, 90, 91 92, 93, 94, 95, 96, 97, 98, 99, or 100 nucleotides in length, or longer). In one embodiment, sgRNA(s) can be between about 15 and about 30 nucleotides in length (e.g., about 15-29, 15-26, 15-25; 16-30, 16-29, 16-26, 16-25; or about 18-30, 18-29, 18-26, or 18-25 nucleotides in length).

To facilitate sgRNA design, many computational tools have been developed (See Prykhozhij et al. (PLoS ONE, 10(3): (2015)); Zhu et al. (PLoS ONE, 9(9) (2014)); Xiao et al. (Bioinformatics. Jan 21 (2014)); Heigwer et al. (Nat Methods, 11(2): 122-123 (2014)). Methods and tools for guide RNA design are discussed by Zhu (Frontiers in Biology, 10 (4) pp 289-296 (2015)), which is incorporated by reference herein. Additionally, there is a publically available software tool that can be used to facilitate the design of sgRNA(s) (http://www.genscript.com/gRNA-design-tool.html).

Inhibitory Nucleic Acids that Hybridize to PHD

In certain embodiments, the PHD inhibitor used in the present methods and compositions is a polynucleotide that reduces expression of PHD. Thus, the method involves administering an effective amount of a polynucleotide that specifically targets nucleotide sequence(s) encoding PHD. The polynucleotides reduce expression of PHD, to yield reduced levels of the gene product (the translated polypeptide).

The nucleic acid target of the polynucleotides (e.g., antisense oligonucleotides, and ribozymes) may be any location within the gene or transcript of PHD.

Any number of means for inhibiting PHD activity or gene expression can be used in the methods of the invention. For example, a nucleic acid molecule complementary to at least a portion of a human PHD encoding nucleic acid can be used to inhibit the gene expression. Means for inhibiting gene expression using short RNA molecules, for example, are known. Among these are short interfering RNA (siRNA), small temporal RNAs (stRNAs), and micro-RNAs (miRNAs). Short interfering RNAs silence genes through an mRNA degradation pathway, while stRNAs and miRNAs are approximately 21 or 22 nt RNAs that are processed from endogenously encoded hairpin-structured precursors, and function to silence genes via translational repression. See, e.g., McManus et al., RNA, 8(6):842-50 (2002); Morris et al., Science, 305(5688):1289-92 (2004); He and Hannon, Nat Rev Genet. 5(7):522-31 (2004).

“RNA interference, or RNAi” is a form of post-transcriptional gene silencing (“PTGS”), and describes effects that result from the introduction of double-stranded RNA into cells (reviewed in Fire, A. Trends Genet 15:358-363 (1999); Sharp, P. Genes Dev 13:139-141 (1999); Hunter, C. Curr Biol 9:R440-R442 (1999); Baulcombe. D. Curr Biol 9:R599-R601 (1999); Vaucheret et al. Plant J 16: 651-659 (1998)). RNA interference, commonly referred to as RNAi, offers a way of specifically inactivating a cloned gene, and is a powerful tool for investigating gene function.

The active agent in RNAi is a long double-stranded (antiparallel duplex) RNA, with one of the strands corresponding or complementary to the RNA which is to be inhibited. The inhibited RNA is the target RNA. The long double stranded RNA is chopped into smaller duplexes of approximately 20 to 25 nucleotide pairs, after which the mechanism by which the smaller RNAs inhibit expression of the target is largely unknown at this time. While RNAi was shown initially to work well in lower eukaryotes, for mammalian cells, it was thought that RNAi might be suitable only for studies on the oocyte and the preimplantation embryo.

More recently, it was shown that RNAi would work in human cells if the RNA strands were provided as pre-sized duplexes of about 19 nucleotide pairs, and RNAi worked particularly well with small unpaired 3′ extensions on the end of each strand (Elbashir et al. Nature 411: 494-498 (2001)). In this report, “short interfering RNA” (siRNA, also referred to as small interfering RNA) were applied to cultured cells by transfection in oligofectamine micelles. These RNA duplexes were too short to elicit sequence-nonspecific responses like apoptosis, yet they efficiently initiated RNAi. Many laboratories then tested the use of siRNA to knock out target genes in mammalian cells. The results demonstrated that siRNA works quite well in most instances.

Software programs for predicting siRNA sequences to inhibit the expression of a target protein are commercially available and find use. One program, siDESIGN from Dharmacon, Inc. (Lafayette, Colo.), permits predicting siRNAs for any nucleic acid sequence, and is available on the internet at dharmacon.com. Programs for designing siRNAs are also available from others, including Genscript (available on the internet at genscript.com/ssl-bin/app/rnai) and, to academic and non-profit researchers, from the Whitehead Institute for Biomedical Research found on the worldwide web at “jura.wi.mit.edu/pubint/http://iona.wi.mit.edu/siRNAext/.”

Any suitable viral knockdown system could be utilized for decreasing PHD mRNA levels—including AAV, lentiviral vectors, or other suitable vectors.

Additionally, specifically targeted delivery of PHD blocking molecule (nucleic acid, peptide, or small molecule) could be delivered by targeted liposome, nanoparticle or other suitable means.

As described herein we provide methods as well as one or more agents/compounds that silence or inhibit PHD, for the treatment, prophylaxis or alleviation of degenerative eye conditions including RP, AMD, glaucoma, and related conditions, as well as neurodegenerative conditions described herein, or predisposition to such conditions.

RNA interference (RNAi) is a method of post transcriptional gene silencing (PTGS) induced by the direct introduction of double-stranded RNA (dsRNA) and has emerged as a useful tool to knock out expression of specific genes in a variety of organisms. RNAi is described by Fire et al., Nature 391:806-811 (1998). Other methods of PTGS are known and include, for example, introduction of a transgene or virus. Generally, in PTGS, the transcript of the silenced gene is synthesized but does not accumulate because it is rapidly degraded. Methods for PTGS, including RNAi are described, for example, in the Ambion.com world wide web site, in the directory “/hottopics/”, in the “rnai” file.

Suitable methods for RNAi in vitro are described herein. One such method involves the introduction of siRNA (small interfering RNA). Current models indicate that these 21-23 nucleotide dsRNAs can induce PTGS. Methods for designing effective siRNAs are described, for example, in the Ambion web site described above. RNA precursors such as Short Hairpin RNAs (shRNAs) can also be encoded by all or a part of the Phd nucleic acid sequence.

Alternatively, double-stranded (ds) RNA is a powerful way of interfering with gene expression in a range of organisms that has recently been shown to be successful in mammals (Wianny and Zernicka-Goetz, 2000, Nat Cell Biol 2:70-75). Double stranded RNA corresponding to the sequence of a Phd polynucleotide can be introduced into or expressed in oocytes and cells of a candidate organism to interfere with Phd activity.

Phd gene expression may also be modulated by introducing peptides or small molecules which inhibit gene expression or functional activity. Thus, compounds identified by the assays described herein as binding to or modulating, such as down-regulating, the amount, activity or expression of PHD polypeptide may be administered to target cells to prevent the function of PHD polypeptide. Such a compound may be administered along with a pharmaceutically acceptable carrier in an amount effective to down-regulate expression or activity PHD, or by activating or down-regulating a second signal which controls PHD expression, activity or amount, and thereby alleviating the abnormal condition.

Alternatively, gene therapy may be employed to control the endogenous production of PHD by the relevant cells such as neuronal cells or photoreceptor cells, i.e., rod and cone cells in the subject. For example, a polynucleotide encoding a PHD siRNA or a portion of this may be engineered for expression in a replication defective retroviral vector, as discussed below. The retroviral expression construct may then be isolated and introduced into a packaging cell transduced with a retroviral plasmid vector containing RNA encoding an anti-Phd siRNA such that the packaging cell now produces infectious viral particles containing the sequence of interest. These producer cells may be administered to a subject for engineering cells in vivo and regulating expression of the PHD polypeptide in vivo. For overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd (1996).

In some embodiments, the level of PHD is decreased in a desired target cell such as a neuronal cell or the vitreous. Furthermore, in such embodiments, treatment may be targeted to, or specific to, desired target cell such as a neuronal cell or the vitreous. The expression of PHD may be specifically decreased only in the desired target cell such as a neuronal cell or the vitreous (i.e., those cells which are predisposed to the condition, or exhibiting the disease already), and not substantially in other non-diseased cells. In these methods, expression of PHD may not be substantially reduced in other cells, i.e., cells which are not desired target cells. Thus, in such embodiments, the level of PHD, remains substantially the same or similar in non-target cells in the course of or following treatment.

Alternately, one may administer the viral vectors, RNAi, shRNA or other PHD inhibitor, or related compounds in a local rather than systemic manner, for example, via injection of directly into the desired target site, often in a depot or sustained release formulation. Furthermore, one may administer the composition in a targeted drug delivery system, for example, in a liposome coated with a tissue-specific antibody, targeting, for example, specific neurons, or the vitreous, and more specifically hepatocytes. The liposomes will be targeted to and taken up selectively by the desired tissue. Also included in a targeted drug delivery system is nanoparticle specific delivery of the viral vectors, RNAi, shRNA or other PHD inhibitors, alone or in combination. A summary of various delivery methods and techniques of siRNA administration in ongoing clinical trials is provided in Zuckerman and Davis 2015; Nature Rev. Drug Discovery, Vol. 14: 843-856, Dec. 2015.

The inhibitory nucleic acids may be an antisense nucleic acid sequence that is complementary to a target region within the mRNA of PHD. The antisense polynucleotide may bind to the target region and inhibit translation. The antisense oligonucleotide may be DNA or RNA, or comprise synthetic analogs of ribo-deoxynucleotides. Thus, the antisense oligonucleotide inhibits expression of PHD.

An antisense oligonucleotide can be, for example, about 7, 10, 15, 20, 25, 30, 35, 40, 45, 50, 55, 60, 65, 70, 75, 80, or more nucleotides in length.

The antisense nucleic acid molecules of the invention may be administered to a subject, or generated in situ such that they hybridize with or bind to the mRNA of PHD.

The administration regimen depends on several factors, including the serum or tissue turnover rate of the therapeutic composition, the level of symptoms, and the accessibility of the target cells in the biological matrix. Preferably, the administration regimen delivers sufficient therapeutic composition to effect improvement in the target disease state, while simultaneously minimizing undesired side effects. Accordingly, the amount of biologic delivered depends in part on the particular therapeutic composition and the severity of the condition being treated.

In certain embodiments, route of administration is subretinal injection or intravitreal injection.

### Ribozyme

The inhibitor may be a ribozyme that inhibits expression of the PHD gene.

Ribozymes can be chemically synthesized and structurally modified to increase their stability and catalytic activity using methods known in the art. Ribozyme encoding nucleotide sequences can be introduced into host cells through gene-delivery mechanisms known in the art.

### Antibodies

The present inhibitors can be an antibody or antigen-binding portion thereof that is specific to PHD.

The antibody or antigen-binding portion thereof may be the following: (a) a whole immunoglobulin molecule; (b) an scFv; (c) a Fab fragment; (d) an F(ab′)2; and (e) a disulfide linked Fv. The antibody or antigen-binding portion thereof may be monoclonal, polyclonal, chimeric and humanized. The antibodies may be murine, rabbit or human/humanized antibodies.

### Combination Therapy

The PHD inhibitor may be administered alone or in combination with a second treatment, such as administration of one or more second agents (different from the present PHD inhibitors), and surgeries.

Non-limiting examples of the second treatments including, treatment with vitamin A, docosahexaenoic acid (DHA), lutein, zeaxanthin, bevacizumab, ranibizumab, pegaptanib, aflibercept, an optic prosthetic device, a gene therapy, a retinal implant (e.g., Argus retinal prosthesis) and/or a retinal sheet transplantation, laser coagulation, photodynamic therapy, and cataract surgery.

The second agents that may be used with the present PHD inhibitor also include, but are not limited to, prostaglandin analogs (e.g., latanoprost, bimatoprost and travoprost), topical beta-adrenergic receptor antagonists (e.g., timolol, levobunolol, and betaxolol), alpha2-adrenergic agonists (e.g., such as brimonidine and apraclonidine), less-selective alpha agonists (e.g., epinephrine), miotic agents (parasympathomimetics, e.g., pilocarpine, and echothiophate), carbonic anhydrase inhibitors (e.g., dorzolamide, brinzolamide, and acetazolamide).

The second treatments that may be used with the present PHD inhibitor include, but are not limited to, laser surgery (e.g., Argon laser trabeculoplasty (ALT), selective laser trabeculoplasty (SLT), Nd:YAG laser peripheral iridotomy (LPI), diode laser cycloablation, traditional laser trabeculoplasty), canaloplasty, trabeculectomy, glaucoma drainage implants, and laser-assisted nonpenetrating deep sclerectomy.

Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

This may be achieved by administering a pharmaceutical composition that includes both agents, or by administering two pharmaceutical compositions, at the same time or within a short time period.

In certain embodiments, the combination of the present PHD inhibitor and the second treatment produces an additive or synergistic effect (i.e., greater than additive effect) in treating a disorder as discussed herein, compared to the effect of the PHD inhibitor alone or the second treatment alone.

As used herein, the term “synergy” (or “synergistic”) means that the effect achieved with the methods and combinations of the combination therapy is greater than the sum of the effects that result from using the individual agents alone, e.g., using the PHD inhibitor alone and the second treatment alone. For example, the effect achieved with the combination of the PHD inhibitor and the second treatment is about 1.1 fold, about 1.2 fold, about 1.3 fold, about 1.4 fold, about 1.5 fold, about 1.6 fold, about 1.7 fold, about 1.8 fold, about 1.9 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 8 fold, about 9 fold, about 10 fold, about 12 fold, about 15 fold, about 20 fold, about 25 fold, about 30 fold, about 50 fold, about 100 fold, at least about 1.2 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, at least about 6 fold, at least about 6.5 fold, at least about 7 fold, at least about 8 fold, at least about 9 fold, at least about 10 fold, of the sum of the effects that result from using the PHD inhibitor alone or the second treatment alone.

Synergistic effects of the combination may also be evidenced by additional, novel effects that do not occur when either agent is administered alone, or by reduction of adverse side effects when either agent is administered alone.

In one embodiment, advantageously, such synergy provides greater efficacy at the same doses, and/or lower side effects.

For sequential administration, either a PHD inhibitor is administered first and then a second treatment, or the second treatment is administered first and then a PHD inhibitor. In embodiments where the PHD inhibitor and the second treatment are administered separately, administration of a first agent can precede administration of a second agent by seconds, minutes, hours, days, or weeks. The time difference in non-simultaneous administrations may be greater than 1 minute, and can be, for example, precisely, at least, up to, or less than 5 minutes, 10 minutes, 15 minutes, 30 minutes, 45 minutes, 60 minutes, 2 hours, 3 hours, 6 hours, 9 hours, 12 hours, 24 hours, 36 hours, or 48 hours, or more than 48 hours. The two or more agents can be administered within minutes of each other or within about 0.5, about 1, about 2, about 3, about 4, about 6, about 9, about 12, about 15, about 18, about 24, or about 36 hours of each other or within about 1, 2, 3, 4, 5, 6, 7, 8, 9, 10, 12, 14 days of each other or within about 2, 3, 4, 5, 6, 7, 8, 9, or 10 weeks of each other. In some cases, longer intervals are possible.

The present disclosure may provide for a pharmaceutical composition comprising a first amount of a PHD inhibitor and a second amount of a second agent. The combination of the first amount of a PHD inhibitor and the second amount of the second agent may produce a synergistic effect on a bacterial infection compared to the effect of the first amount of the PHD inhibitor alone or the effect of the second amount of the second agent alone.

The amount of a PHD inhibitor or the amount of the second agent that may be used in the combination therapy may be a therapeutically effective amount, a sub-therapeutically effective amount or a synergistically effective amount.

The PHD inhibitor, and/or the second agent may be present in the pharmaceutical composition in an amount ranging from about 0.005% (w/w) to about 100% (w/w), from about 0.01% (w/w) to about 90% (w/w), from about 0.1% (w/w) to about 80% (w/w), from about 1% (w/w) to about 70% (w/w), from about 10% (w/w) to about 60% (w/w), from about 0.01% (w/w) to about 15% (w/w), or from about 0.1% (w/w) to about 20% (w/w).

The PHD inhibitor and the second agent may be present in two separate pharmaceutical compositions to be used in a combination therapy.

The effective amount of the PHD inhibitor or the second agent for the combination therapy may be less than, equal to, or greater than when the agent is used alone.

### Pharmaceutical Compositions

The present agents or pharmaceutical compositions may be administered by any route, including, without limitation, oral, transdermal, ocular, intraperitoneal, intravenous, ICV, intracisternal injection or infusion, subcutaneous, implant, sublingual, subcutaneous, intramuscular, intravenous, rectal, mucosal, ophthalmic, intrathecal, intra-articular, intra-arterial, sub-arachinoid, bronchial and lymphatic administration. The present composition may be administered parenterally or systemically.

The pharmaceutical compositions of the present invention can be, e.g., in a solid, semi-solid, or liquid formulation. Intranasal formulation can be delivered as a spray or in a drop; inhalation formulation can be delivered using a nebulizer or similar device; topical formulation may be in the form of gel, ointment, paste, lotion, cream, poultice, cataplasm, plaster, dermal patch aerosol, etc.; transdermal formulation may be administered via a transdermal patch or iontorphoresis. Compositions can also take the form of tablets, pills, capsules, semisolids, powders, sustained release formulations, solutions, emulsions, suspensions, elixirs, aerosols, chewing bars or any other appropriate compositions.

The composition may be administered locally via implantation of a membrane, sponge, or another appropriate material on to which the desired molecule has been absorbed or encapsulated. Where an implantation device is used, the device may be implanted into any suitable tissue or organ, and delivery of the desired molecule may be via diffusion, timed release bolus, or continuous administration.

To prepare such pharmaceutical compositions, one or more of compound of the present invention may be mixed with a pharmaceutical acceptable excipient, e.g., a carrier, adjuvant and/or diluent, according to conventional pharmaceutical compounding techniques.

Pharmaceutically acceptable carriers that can be used in the present compositions encompass any of the standard pharmaceutical carriers, such as a phosphate buffered saline solution, water, and emulsions, such as an oil/water or water/oil emulsion, and various types of wetting agents. The compositions can additionally contain solid pharmaceutical excipients such as starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols. For examples of carriers, stabilizers, preservatives and adjuvants, see Remington's Pharmaceutical Sciences, edited by E. W. Martin (Mack Publishing Company, 18th ed., 1990). Additional excipients, for example sweetening, flavoring and coloring agents, may also be present.

The pharmaceutically acceptable excipient may be selected from the group consisting of fillers, e.g. sugars and/or sugar alcohols, e.g. lactose, sorbitol, mannitol, maltodextrin, etc.; surfactants, e.g. sodium lauryle sulfate, Brij 96 or Tween 80; disintegrants, e.g. sodium starch glycolate, maize starch or derivatives thereof; binder, e.g. povidone, crosspovidone, polyvinylalcohols, hydroxypropylmethylcellulose; lubricants, e.g. stearic acid or its salts; flowability enhancers, e.g. silicium dioxide; sweeteners, e.g. aspartame; and/or colorants. Pharmaceutically acceptable carriers include any and all clinically useful solvents, dispersion media, coatings, antibacterial and antifungal agents, isotonic and absorption delaying agents and the like.

The pharmaceutical composition may contain excipients for modifying, maintaining or preserving, for example, the pH, osmolarity, viscosity, clarity, color, isotonicity, odor, sterility, stability, rate of dissolution or release, adsorption or penetration of the composition. Suitable excipients include, but are not limited to, amino acids (such as glycine, glutamine, asparagine, arginine or lysine); antimicrobials; antioxidants (such as ascorbic acid, sodium sulfite or sodium hydrogen sulfite); buffers (such as borate, bicarbonate, Tris HCl, citrates, phosphates, other organic acids); bulking agents (such as mannitol or glycine), chelating agents (such as ethylenediamine tetraacetic acid (EDTA), ethylene glycol tetraacetic acid (EGTA)); complexing agents (such as caffeine, polyvinylpyrrolidone, beta cyclodextrin or hydroxypropyl beta cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (in one aspect, sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company, 1990).

Oral dosage forms may be tablets, capsules, bars, sachets, granules, syrups and aqueous or oily suspensions. Tablets may be formed form a mixture of the active compounds with fillers, for example calcium phosphate; disintegrating agents, for example maize starch, lubricating agents, for example magnesium stearate; binders, for example microcrystalline cellulose or polyvinylpyrrolidone and other optional ingredients known in the art to permit tabletting the mixture by known methods. Similarly, capsules, for example hard or soft gelatin capsules, containing the active compound, may be prepared by known methods. The contents of the capsule may be formulated using known methods so as to give sustained release of the active compounds. Other dosage forms for oral administration include, for example, aqueous suspensions containing the active compounds in an aqueous medium in the presence of a non-toxic suspending agent such as sodium carboxymethylcellulose, and oily suspensions containing the active compounds in a suitable vegetable oil, for example arachis oil. The active compounds may be formulated into granules with or without additional excipients. The granules may be ingested directly by the patient or they may be added to a suitable liquid carrier (e.g. water) before ingestion. The granules may contain disintegrants, e.g. an effervescent pair formed from an acid and a carbonate or bicarbonate salt to facilitate dispersion in the liquid medium. U.S. Pat. No. 8,263,662.

Intravenous forms include, but are not limited to, bolus and drip injections. Examples of intravenous dosage forms include, but are not limited to, Water for Injection USP; aqueous vehicles including, but not limited to, Sodium Chloride Injection, Ringer's Injection, Dextrose Injection, Dextrose and Sodium Chloride Injection, and Lactated Ringer's Injection; water-miscible vehicles including, but not limited to, ethyl alcohol, polyethylene glycol and polypropylene glycol; and non-aqueous vehicles including, but not limited to, corn oil, cottonseed oil, peanut oil, sesame oil, ethyl oleate, isopropyl myristate and benzyl benzoate.

Additional compositions include formulations in sustained or controlled delivery, such as using liposome or micelle carriers, bioerodible microparticles or porous beads and depot injections.

The present compound(s) or composition may be administered as a single dose, or as two or more doses (which may or may not contain the same amount of the desired molecule) over time, or as a continuous infusion via implantation device or catheter. The pharmaceutical composition can be prepared in single unit dosage forms.

Appropriate frequency of administration can be determined by one of skill in the art and can be administered once or several times per day (e.g., twice, three, four or five times daily). The compositions of the invention may also be administered once each day or once every other day. The compositions may also be given twice weekly, weekly, monthly, or semi-annually. In the case of acute administration, treatment is typically carried out for periods of hours or days, while chronic treatment can be carried out for weeks, months, or even years. U.S. Pat. No. 8,501,686.

Administration of the compositions of the invention can be carried out using any of several standard methods including, but not limited to, continuous infusion, bolus injection, intermittent infusion, inhalation, or combinations of these methods. For example, one mode of administration that can be used involves continuous intravenous infusion. The infusion of the compositions of the invention can, if desired, be preceded by a bolus injection.

The amount of the PHD inhibitor (e.g., a first amount) or the amount of the second agent (e.g., a second amount) that may be used in the combination therapy may be a therapeutically effective amount, a sub-therapeutically effective amount or a synergistically effective amount. The amounts are dosages that achieve the desired synergism.

As used herein, the term “therapeutically effective amount” is an amount sufficient to treat a specified disorder or disease or alternatively to obtain a pharmacological response treating a disorder or disease.

Methods of determining the most effective means and dosage of administration can vary with the composition used for therapy, the purpose of the therapy, the target cell being treated, and the subject being treated. Single or multiple administrations can be carried out with the dose level and pattern being selected by the treating physician. The specific dose level for any particular subject depends upon a variety of factors including the activity of the specific peptide, the age, body weight, general health, sex, diet, time of administration, route of administration, and rate of excretion, drug combination and the severity of the particular disease undergoing therapy.

For example, the present PHD inhibitor may be administered at about 0.0001 mg/kg to about 500 mg/kg, about 0.01 mg/kg to about 200 mg/kg, about 0.01 mg/kg to about 0.1 mg/kg, about 0.1 mg/kg to about 100 mg/kg, about 10 mg/kg to about 200 mg/kg, about 10 mg/kg to about 20 mg/kg, about 5 mg/kg to about 15 mg/kg, about 0.0001 mg/kg to about 0.001 mg/kg, about 0.001 mg/kg to about 0.01 mg/kg, about 0.01 mg/kg to about 0.1 mg/kg, about 0.1 mg/kg to about 0.5 mg/kg, about 0.5 mg/kg to about 1 mg/kg, about 1 mg/kg to about 2.5 mg/kg, about 2.5 mg/kg to about 10 mg/kg, about 10 mg/kg to about 50 mg/kg, about 50 mg/kg to about 100 mg/kg, about 100 mg/kg to about 250 mg/kg, about 0.1 μg/kg to about 800 μg/kg, about 0.5 μg/kg to about 500 μg/kg, about 1 μg/kg to about 20 μg/kg, about 1 μg/kg to about 10 μg/kg, about 10 μg/kg to about 20 μg/kg, about 20 μg/kg to about 40 μg/kg, about 40 μg/kg to about 60 μg/kg, about 60 μg/kg to about 100 μg/kg, about 100 μg/kg to about 200 μg/kg, about 200 μg/kg to about 300 μg/kg, or about 400 μg/kg to about 600 μg/kg. In some embodiments, the dose is within the range of about 250 mg/kg to about 500 mg/kg, about 0.5 mg/kg to about 50 mg/kg, or any other suitable amounts.

In certain embodiments, the amount or dose of the present PHD inhibitor may range from about 0.01 mg to about 10 g, from about 0.1 mg to about 9 g, from about 1 mg to about 8 g, from about 1 mg to about 7 g, from about 5 mg to about 6 g, from about 10 mg to about 5 g, from about 20 mg to about 1 g, from about 50 mg to about 800 mg, from about 100 mg to about 500 mg, from about 600 mg to about 800 mg, from about 800 mg to about 1 g, from about 0.01mg to about 10 g, from about 0.05 μg to about 1.5 mg, from about 10 μg to about 1 mg protein, from about 0.1 mg to about 10 mg, from about 2 mg to about 5 mg, from about 1 mg to about 20 mg, from about 30 μg to about 500 μg, from about 40 pg to about 300 pg, from about 0.1 μg to about 200 mg, from about 0.1 μg to about 5 μg, from about 5 μg to about 10 μg, from about 10 μg to about 25 μg, from about 25 μg to about 50 μg, from about 50 μg to about 100 μg, from about 100 μg to about 500 μg, from about 500 μg to about 1 mg, from about 1 mg to about 2 mg, e.g., in the pharmaceutical composition.

The dose of the present PHD inhibitor may range from about 0.1 μg/day to about 1 mg/day, from about 10 μg/day to about 200 μg/day, from about 20 μg/day to about 150 μg/day, from about 0.1 μg/day to about 125 μg/day, from about 1 μg/day to about 20 μg/day, or about 4.5 μg/day to about 30 μg/day.

Different dosage regimens may be used. In some embodiments, a daily dosage, such as any of the exemplary dosages described above, is administered once, twice, three times, or four times a day for at least three, four, five, six, seven, eight, nine, or ten days. Depending on the stage and severity of the cancer, a shorter treatment time (e.g., up to five days) may be employed along with a high dosage, or a longer treatment time (e.g., ten or more days, or weeks, or a month, or longer) may be employed along with a low dosage. In some embodiments, a once- or twice-daily dosage is administered every other day.

### Kits

In another aspect, the present invention provides any of the compositions described herein in kits, optionally including instructions for use of the compositions (e.g., for improving neuronal survival and/or inhibiting PHD). That is, the kit can include a description of use of a composition in any method described herein. A “kit,” as used herein, typically defines a package, assembly, or container (such as an insulated container) including one or more of the components or embodiments of the invention, and/or other components associated with the invention, for example, as previously described. Each of the components of the kit may be provided in liquid form (e.g., in solution), or in solid form (e.g., a dried powder, frozen, etc.).

In some cases, the kit includes one or more components, which may be within the same or in two or more receptacles, and/or in any combination thereof. The receptacle is able to contain a liquid, and non-limiting examples include bottles, vials, jars, tubes, flasks, beakers, or the like. In some cases, the receptacle is spill-proof (when closed, liquid cannot exit the receptacle, regardless of orientation of the receptacle).

Examples of other compositions or components associated with the invention include, but are not limited to, diluents, salts, buffers, chelating agents, preservatives, drying agents, antimicrobials, needles, syringes, packaging materials, tubes, bottles, flasks, beakers, and the like, for example, for using, modifying, assembling, storing, packaging, preparing, mixing, diluting, and/or preserving the components for a particular use. In embodiments where liquid forms of any of the components are used, the liquid form may be concentrated or ready to use.

A kit of the invention generally will include instructions or instructions to a website or other source in any form that are provided for using the kit in connection with the components and/or methods of the invention. For instance, the instructions may include instructions for the use, modification, mixing, diluting, preserving, assembly, storage, packaging, and/or preparation of the components and/or other components associated with the kit. In some cases, the instructions may also include instructions for the delivery of the components, for example, for shipping at room temperature, sub-zero temperatures, cryogenic temperatures, etc. The instructions may be provided in any form that is useful to the user of the kit, such as written or oral (e.g., telephonic), digital, optical, visual (e.g., videotape, DVD, etc.) and/or electronic communications (including Internet or web-based communications), provided in any manner.

As used herein, instructions can include protocols, directions, guides, warnings, labels, notes, and/or “frequently asked questions” (FAQs), and typically involve written instructions on or associated with the invention and/or with the packaging of the invention. Instructions can also include instructional communications in any form (e.g., oral, electronic, digital, optical, visual, etc.), provided in any manner (e.g., within or separate from a kit) such that a user will clearly recognize that the instructions are to be used with the kit.

The following are examples of the present invention and are not to be construed as limiting.

### Example 1

While more than 60 genes are implicated in RP, mutations in the genes encoding the phosphodiesterase 6 (PDE6) are responsible for 72,000 new cases of RP worldwide. This gene encodes a rod-specific protein that regulates cyclic guanosine monophosphate (cGMP) levels and consequently plays a critical role in normal phototransduction. Mutations in PDE6 provoke a metabolic imbalance between catabolism and anabolism in rods that forces them to succumb to the consequences of elevated cGMP and Ca²⁺.

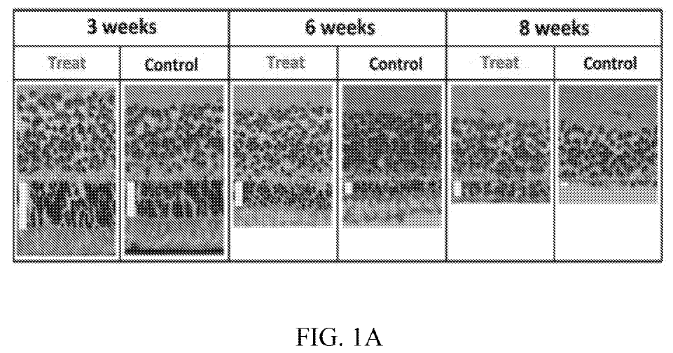

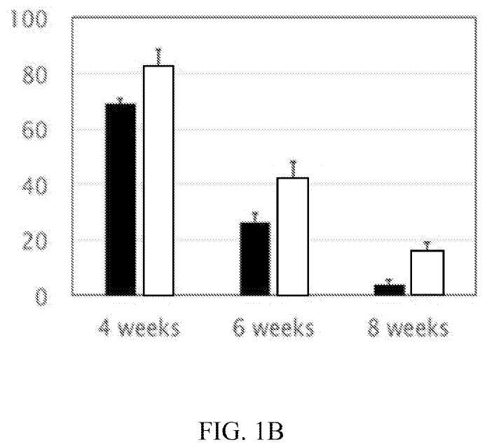

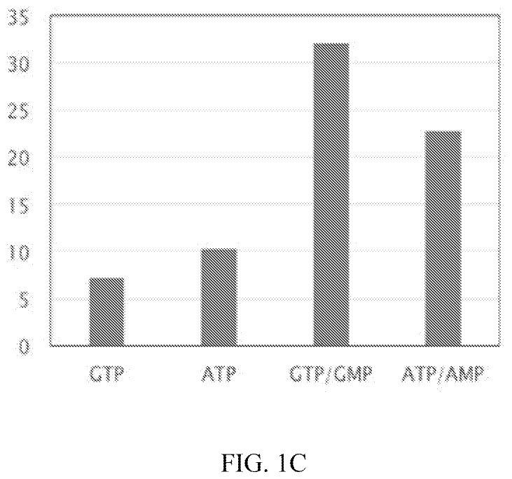

We used an inducible, floxed-CreER, Pde6b mouse model of RP to genetically enhance glycolysis in photoreceptors and found that the balance between catabolic and anabolic metabolism was improved, which slowed photoreceptor degeneration and prevented subsequent vision loss (see FIGS. 1A-1C). In this study, we aim to develop a more translational and clinically relevant therapy.

Enhancing the natural ability of photoreceptors to take up and incorporate nutrients into biomass (i.e., anabolism) could improve their resistance to degeneration (see FIGS. 1A-1C).

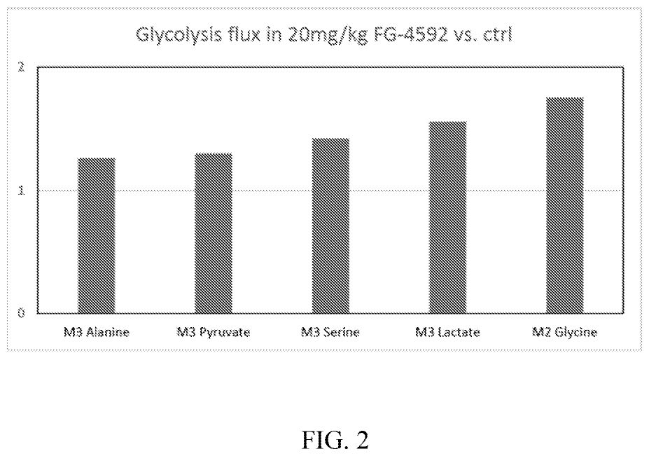

Our data show PHD and a-ketoglutarate downregulate glycolysis. We administered a PHD antagonist to a RP mouse and found that results were comparable to those we previously obtained (see FIG. 2) with a floxed-CreER system data.

We will determine whether PHD antagonists are effective at increasing glycolysis and rescuing vision in different retinal degenerative backgrounds. PHD antagonists will be administered to Pde6bH620Q/H620Q, RhoD190N, and Pde6aD670G/D670G mice to determine whether PHD is effective regardless of genetic background.

We will compare the efficacy of a variety of PHD inhibitors and optimize the dosage protocol. A number of PHD inhibitors, such as FG-4592 (NCT02021318), BAY 85-3934 (Molidustat), JNJ-42041935, Hydralazine hydrochloride, and any inhibitors described herein, will be tested.

The dosage range will be optimized by varying the dosage amount and timing of the PHD inhibitor and efficacy of rescue assessed. All efficacy outcomes will be assessed by ERG, histology, and ¹³C glucose tracings.

### REFERENCES

Zhang et al., “Reprogramming toward anabolism impedes degeneration in a preclinical model of retinitis pigment, Human Molecular Genetics 2016. PMID: 27516389.

Zhang et al., Reprogramming metabolism by targeting sirtuin 6 attenuates retinal degeneration. J. Clin. Invest. 2016 Nov 14. pii: 86905. doi: 10.1172/JCI86905. PMID:27841758

Li et al., Long-term safety and efficacy of human induced pluripotent stem cell (iPS) grafts in a preclinical model of retinitis pigmentosa, (2012) Molecular Medicine. 18:1312-9.

Wert et al., Gene therapy provides long-term visual function in a pre-clinical model of retinitis pigmentosa, (2013) Human Molecular Genetics. 22:558-567.

Davis et al., Therapeutic margins in a novel preclinical model of retinitis pigmentosa, (2013) Journal of Neuroscience. 33:13475-83.

Koch et al., Halting progressive neurodegeneration in advanced retinitis pigmentosa. J Clin Invest. 2015 Sep 1;125(9):3704-13.

Yang et al., (2014) Human Molecular Genetics. Jan 31. PMID: 24497574.

Li et al., Gene therapy in patient-specific stem cell lines and a preclinical model of retinitis pigmentosa with membrane frizzled related protein (MFRP) defects, (2014) Molecular Therapy. 2014 Sep; 22(9):1688-97.

Tsang et al., Retinal Degeneration in Mice Lacking the y subunit of cGMP phosphodiesterase, (1996) Science 272: 1026-1029.

Tsang et al., Role of the Target Enzyme in Deactivation of Photoreceptor G Protein in Vivo, (1998) Science. 282, 117-21.

Tsang et al., Modulation of phosphodiesterase6 turnoff during background illumination in mouse rod photoreceptors (2006). J. Neurosci. 26, 4472-4480.

Yau KW. Phototransduction mechanism in retinal rods and cones. The Friedenwald Lecture. Investigative ophthalmology & visual science. 1994;35(1):9-32. Zwaans BM, and Lombard DB. Interplay between sirtuins, MYC and hypoxia-inducible factor in cancer-associated metabolic reprogramming. Dis Model Mech. 2014;7(9):1023-32.

Mohand-Said S, Hicks D, Dreyfus H, and Sahel JA. Selective transplantation of rods delays cone loss in a retinitis pigmentosa model. Archives of ophthalmology. 2000;118(6):807-11.

Xiong W, MacColl Garfinkel AE, Li Y, Benowitz LI, and Cepko CL. NRF2 promotes neuronal survival in neurodegeneration and acute nerve damage. The Journal of clinical investigation. 2015; 125(4): 1433-45.

Cepko C, and Punzo C. Cell metabolism: Sugar for sight. Nature. 2015;522(7557):428-9.

Cideciyan A V, Jacobson S G, Beltran W A, Sumaroka A, Swider M, Iwabe S, Roman A J, Olivares M B, Schwartz S B, Komaromy A M, et al. Human retinal gene therapy for Leber congenital amaurosis shows advancing retinal degeneration despite enduring visual improvement. Proceedings of the National Academy of Sciences of the United States of America. 2013; 110(6):E517-25.

Jacobson S G, Cideciyan A V, Roman A J, Sumaroka A, Schwartz S B, Heon E, and Hauswirth W W. Improvement and decline in vision with gene therapy in childhood blindness. The New England journal of medicine. 2015; 372(20):1920-6.

Wright A F. Long-term effects of retinal gene therapy in childhood blindness. The New England journal of medicine. 2015; 372(20):1954-5.

Du J, Rountree A, Cleghorn W M, Contreras L, Lindsay K J, Sadilek M, Gu H, Djukovic D, Raftery D, Satrustegui J, et al. Phototransduction Influences Metabolic Flux and Nucleotide Metabolism in Mouse Retina. The Journal of biological chemistry. 2016; 291(9):4698-710.

Demetrius L A, Magistretti P J, and Pellerin L. Alzheimer's disease: the amyloid hypothesis and the Inverse Warburg effect. Front Physiol. 2014; 5(522.

Demetrius L A, and Simon D K. An inverse-Warburg effect and the origin of Alzheimer's disease. Biogerontology. 2012; 13(6):583-94.

Mariani A P. Neuronal and synaptic organization of the outer plexiform layer of the pigeon retina. The American journal of anatomy. 1987; 179(1):25-39.

Masland R H. The neuronal organization of the retina. Neuron. 2012; 76(2):266-80.

Kim H S, Xiao C, Wang R H, Lahusen T, Xu X, Vassilopoulos A, Vazquez-Ortiz G, Jeong W I, Park O, Ki S H, et al. Hepatic-specific disruption of SIRT6 in mice results in fatty liver formation due to enhanced glycolysis and triglyceride synthesis. Cell metabolism. 2010; 12(3):224-36.

Hart A W, McKie L, Morgan J E, Gautier P, West K, Jackson I J, and Cross S H. Genotype-phenotype correlation of mouse pde6b mutations. Investigative ophthalmology & visual science. 2005; 46(9):3443-50.

Koch SF, Tsai YT, Duong J K, Wu W H, Hsu C W, Wu W P, Bonet-Ponce L, Lin C S, and Tsang S H. Halting progressive neurodegeneration in advanced retinitis pigmentosa. The Journal of clinical investigation. 2015; 125(9):3704-13.

Tsang S H, Chen J, Kjeldbye H, Li W S, Simon M I, Gouras P, and Goff S P. Retarding photoreceptor degeneration in Pdegtml/Pdegtml mice by an apoptosis suppressor gene. Investigative ophthalmology & visual science. 1997; 38(5):943-50.

Tanabe T, Tsang SH, Kjeldbye H, Berns K, Goff S, and Gouras P. Adeno-associated virus mediated gene transfer into pde g knockout mouse. Investigative ophthalmology & visual science. 1998:S5153.

Tosi J, Davis R J, Wang N K, Naumann M, Lin C S, and Tsang S H. shRNA knockdown of guanylate cyclase 2e or cyclic nucleotide gated channel alpha 1 increases photoreceptor survival in a cGMP phosphodiesterase mouse model of retinitis pigmentosa. J Cell Mol Med. 2011; 15(8):1778-87.

Tsang S H, Tsui I, Chou C L, Zernant J, Haamer E, Iranmanesh R, Tosi J, and Allikmets R. A novel mutation and phenotypes in phosphodiesterase 6 deficiency. American journal of ophthalmology. 2008; 146(5):780-8.

Tsang S, Woodruff M, Lin C, Jacobson B, Naumann M, Hsu C, Davis R, Cilluffo M C, Chen J, and Fain G. Effect of the ILE86TER mutation in the γ subunit of cGMP phosphodiesterase (PDE6) on rod photoreceptor signalling. Cellular Signalling. 2012; 0(0.

Du J, Linton J D, and Hurley J B. Probing Metabolism in the Intact Retina Using Stable Isotope Tracers. Methods in enzymology. 2015; 561(149-70.

Du J, Cleghorn W, Contreras L, Linton J D, Chan G C, Chertov A O, Saheki T, Govindaraju V, Sadilek M, Satrustegui J, et al. Cytosolic reducing power preserves glutamate in retina. Proceedings of the National Academy of Sciences of the United States of America. 2013; 110(46):18501-6.

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description is offered by way of illustration only and not as a limitation.

TABLE 1
Hypoxia-Inducible Factor-Prolyl Hydroxylase (HIF-PH) Inhibitors

| | | | | | Sub- | | | | | |
|---|---|---|---|---|---|---|---|---|---|---|
| | | | | Pub- | stance | | | EphMRA | Chemical/ | |
| Product | Other | Active | CAS | Chem | of | | | Drug | Biological | Product |
| Name | Names | Ingredient | No. | ID | Origin | Target | MOA | Class | Class | Description |
| AKB6548 | AKB6548, | vadadustat | 1000025- | — | — | Hypoxia- | Hypoxia- | anti- | Pyridines | AKB6548 |
| | AKB6548 | | 07-9 | | | Inducible | Inducible | anaemic | | contains |
| | | | | | | Factor- | Factor- | preparations | | vadadustat as |
| | | | | | | Prolyl | Prolyl | | | an active |
| | | | | | | Hydroxy- | Hydroxy- | | | ingredient. |
| | | | | | | lase | lase | | | Vadadustat is |
| | | | | | | (HIF-PH) | (HIF-PH) | | | a small |
| | | | | | | | Inhibitor | | | molecule HIF- |
| | | | | | | | | | | PH (hypoxia- |
| | | | | | | | | | | inducible |
| | | | | | | | | | | factor-prolyl |
| | | | | | | | | | | hydroxylase) |
| | | | | | | | | | | inhibitor |
| | | | | | | | | | | designed to |
| | | | | | | | | | | increase the |
| | | | | | | | | | | natural |
| | | | | | | | | | | production of |
| | | | | | | | | | | erythropoietin |
| | | | | | | | | | | and cause a |
| | | | | | | | | | | controlled, |
| | | | | | | | | | | gradual rise in |
| | | | | | | | | | | hemoglobin in |
| | | | | | | | | | | anemia. |
| | | | | | | | | | | AKB6548 is |
| | | | | | | | | | | indicated for |
| | | | | | | | | | | the treatment |
| | | | | | | | | | | of anemia |
| | | | | | | | | | | associated |
| | | | | | | | | | | with chronic |
| | | | | | | | | | | kidney disease |
| | | | | | | | | | | (CKD). |
| AKB6899 | AKB6899 | — | — | — | — | Hypoxia- | Hypoxia- | All other | — | AKB6899 is |
| | | | | | | Inducible | Inducible | antineo- | | an orally |
| | | | | | | Factor- | Factor- | plastics, | | bioavailable |
| | | | | | | Prolyl | Prolyl | other | | small |
| | | | | | | Hydroxy- | Hydroxy- | ophthalmo- | | molecule HIF- |
| | | | | | | lase | lase | logicals | | PH (hypoxia- |
| | | | | | | (HIF-PH) | (HIF-PH) | | | inducible |
| | | | | | | | Inhibitor | | | factor-prolyl |
| | | | | | | | | | | hydroxylase) |
| | | | | | | | | | | inhibitor. It |
| | | | | | | | | | | inhibits the |
| | | | | | | | | | | enzyme |
| | | | | | | | | | | hypoxia |
| | | | | | | | | | | inducible |
| | | | | | | | | | | factor-propyl |
| | | | | | | | | | | hydroxylase |
| | | | | | | | | | | that regulates |
| | | | | | | | | | | the stability |
| | | | | | | | | | | and activity of |
| | | | | | | | | | | HIF (hypoxia- |
| | | | | | | | | | | inducible |
| | | | | | | | | | | factor). It |
| | | | | | | | | | | selectively |
| | | | | | | | | | | activates the |
| | | | | | | | | | | body’s natural |
| | | | | | | | | | | process of |
| | | | | | | | | | | HIF-2- |
| | | | | | | | | | | mediated |
| | | | | | | | | | | erythropoiesis, |
| | | | | | | | | | | including the |
| | | | | | | | | | | induction of |
| | | | | | | | | | | endogenous |
| | | | | | | | | | | EPO, the |
| | | | | | | | | | | mobilization |
| | | | | | | | | | | and utilization |
| | | | | | | | | | | of iron stores, |
| | | | | | | | | | | and |
| | | | | | | | | | | suppression of |
| | | | | | | | | | | inflammatory |
| | | | | | | | | | | cytokines |
| | | | | | | | | | | essential to the |
| | | | | | | | | | | formation of |
| | | | | | | | | | | new oxygen- |
| | | | | | | | | | | carrying red |
| | | | | | | | | | | blood cell. It |
| | | | | | | | | | | significantly |
| | | | | | | | | | | inhibits HIF- |
| | | | | | | | | | | PH dependent |
| | | | | | | | | | | expression of |
| | | | | | | | | | | vascular |
| | | | | | | | | | | endothelial |
| | | | | | | | | | | growth factor |
| | | | | | | | | | | (VEGF) and |
| | | | | | | | | | | phosphogly- |
| | | | | | | | | | | cerate kinase |
| | | | | | | | | | | (PGK) and |
| | | | | | | | | | | stimulates the |
| | | | | | | | | | | production of |
| | | | | | | | | | | soluble |
| | | | | | | | | | | vascular |
| | | | | | | | | | | endothelial |
| | | | | | | | | | | growth factor |
| | | | | | | | | | | receptor l |
| | | | | | | | | | | (sVEGFr1). |
| | | | | | | | | | | AKB6899 is |
| | | | | | | | | | | indicated for |
| | | | | | | | | | | the treatment |
| | | | | | | | | | | of VEGF |
| | | | | | | | | | | (vascular |
| | | | | | | | | | | endothelial |
| | | | | | | | | | | growth factor |
| | | | | | | | | | | receptor) |
| | | | | | | | | | | related eye |
| | | | | | | | | | | diseases and |
| | | | | | | | | | | solid tumors. |
| BAY853934 | BAY853934, | molidustat | 1154028- | 59603622 | Synthetic | Hypoxia- | Hypoxia- | anti- | Morpholines, | BAY853934 |
| | BAY853934 | | 82-6 | | | Inducible | Inducible | anaemic | Pyrazoles, | contains |
| | | | | | | Factor- | Factor- | preparations | Pyrimidines, | molidustat as |
| | | | | | | Prolyl | Prolyl | | Triazoles | an active |
| | | | | | | Hydroxy- | Hydroxy- | | | ingredient. It |
| | | | | | | lase | lase | | | is a hypoxia |
| | | | | | | (HIF-PH) | (HIF-PH) | | | inducible |
| | | | | | | | Inhibitor | | | factor prolyl |
| | | | | | | | | | | hydroxylase |
| | | | | | | | | | | (HIF-PH) |
| | | | | | | | | | | Inhibitor. HIF- |
| | | | | | | | | | | PH Inhibitor |
| | | | | | | | | | | inhibits the |
| | | | | | | | | | | enzyme |
| | | | | | | | | | | hypoxia |
| | | | | | | | | | | inducible |
| | | | | | | | | | | factor-propyl |
| | | | | | | | | | | hydroxylase |
| | | | | | | | | | | which |
| | | | | | | | | | | stimulates |
| | | | | | | | | | | erythropoietin |
| | | | | | | | | | | (EPO) |
| | | | | | | | | | | production |
| | | | | | | | | | | and the |
| | | | | | | | | | | formation of |
| | | | | | | | | | | red blood |
| | | | | | | | | | | cells. |
| | | | | | | | | | | BAY853934 |
| | | | | | | | | | | is indicated |
| | | | | | | | | | | for the |
| | | | | | | | | | | treatment of |
| | | | | | | | | | | renal anemia |
| | | | | | | | | | | due to chronic |
| | | | | | | | | | | kidney |
| | | | | | | | | | | disease. |
| DS1093 | DS1093, | — | — | — | — | Hypoxia- | Hypoxia- | other anti- | — | DS1093 is a |
| | DS1093 a, | | | | | Inducible | Inducible | anaemic | | small |
| | DS1093a, | | | | | Factor- | Factor- | products, | | molecule |
| | DS1093 a, | | | | | Prolyl | Prolyl | including | | which acts as |
| | DS1093a | | | | | Hydroxy- | Hydroxy- | folic acid, | | hypoxia- |
| | | | | | | lase | lase | folinic acid | | inducible |
| | | | | | | (HIF-PH) | (HIF-PH) | | | factor-prolyl |
| | | | | | | | Inhibitor | | | hydroxylase |
| | | | | | | | | | | (HIF-PH) |
| | | | | | | | | | | inhibitor. |
| | | | | | | | | | | DS 1093 is |
| | | | | | | | | | | indicated for |
| | | | | | | | | | | the treatment |
| | | | | | | | | | | of anemia |
| | | | | | | | | | | associated |
| | | | | | | | | | | with chronic |
| | | | | | | | | | | kidney |
| | | | | | | | | | | disease. |
| FG2216 | FG2216, | FG2216 | 223387- | 6914666 | Synthetic | Hypoxia- | Hypoxia- | erythro- | Isoquino- | FG2216 is an |
| | FG2216, | | 75-5 | | | Inducible | Inducible | poietin | lines | inhibitor of |
| | YM311, | | | | | Factor- | Factor- | products | | hypoxia- |
| | YM311 | | | | | Prolyl | Prolyl | | | inducible |
| | | | | | | Hydroxy- | Hydroxy- | | | factor-prolyl |
| | | | | | | lase | lase | | | hydroxylase |
| | | | | | | (HIF-PH) | (HIF-PH) | | | (HIF-PH), an |
| | | | | | | | Inhibitor | | | enzyme that |
| | | | | | | | | | | regulates the |
| | | | | | | | | | | stability and |
| | | | | | | | | | | activity of |
| | | | | | | | | | | HIF. It |
| | | | | | | | | | | selectively |
| | | | | | | | | | | activates the |
| | | | | | | | | | | body’s natural |
| | | | | | | | | | | process of |
| | | | | | | | | | | HIF-2- |
| | | | | | | | | | | mediated |
| | | | | | | | | | | erythropoiesis, |
| | | | | | | | | | | including the |
| | | | | | | | | | | induction of |
| | | | | | | | | | | endogenous |
| | | | | | | | | | | EPO, the |
| | | | | | | | | | | mobilization |
| | | | | | | | | | | and utilization |
| | | | | | | | | | | of iron stores, |
| | | | | | | | | | | and |
| | | | | | | | | | | suppression of |
| | | | | | | | | | | inflammatory |
| | | | | | | | | | | cytokines |
| | | | | | | | | | | essential to the |
| | | | | | | | | | | formation of |
| | | | | | | | | | | new oxygen- |
| | | | | | | | | | | carrying red |
| | | | | | | | | | | blood cells. |
| | | | | | | | | | | FG2216 is |
| | | | | | | | | | | indicated for |
| | | | | | | | | | | the treatment |
| | | | | | | | | | | of anemia |
| | | | | | | | | | | associated |
| | | | | | | | | | | with chronic |
| | | | | | | | | | | renal failure |
| | | | | | | | | | | and |
| | | | | | | | | | | chemotherapy |
| | | | | | | | | | | induced |
| | | | | | | | | | | anemia. |
| FG4497 | FG4497 | — | — | — | — | Hypoxia- | Hypoxia- | all other | — | PG-4497 is a |
| | | | | | | Inducible | Inducible | urological | | hypoxia- |
| | | | | | | Factor- | Factor- | products | | inducible |
| | | | | | | Prolyl | Prolyl | | | factor (HIF) |
| | | | | | | Hydroxy- | Hydroxy- | | | prolyl |
| | | | | | | lase | lase | | | hydroxylase- |
| | | | | | | (HIF-PH) | (HIF-PH) | | | (PH) inhibitor |
| | | | | | | | Inhibitor | | | that activates |
| | | | | | | | | | | hypoxia- |
| | | | | | | | | | | inducible |
| | | | | | | | | | | factor. FG- |
| | | | | | | | | | | 4497 is |
| | | | | | | | | | | indicated for |
| | | | | | | | | | | the protection |
| | | | | | | | | | | of kidney |
| | | | | | | | | | | from acute |
| | | | | | | | | | | and chronic |
| | | | | | | | | | | injury. |
| FG4539 | FG4539 | — | — | — | — | Hypoxia- | Hypoxia- | all other | — | FG4539 is a |
| | | | | | | Inducible | Inducible | CNS drugs, | | novel small |
| | | | | | | Factor- | Factor- | all other | | molecule that |
| | | | | | | Prolyl | Prolyl | urological | | acts as a |
| | | | | | | Hydroxy- | Hydroxy- | products, | | cytoprotectant, |
| | | | | | | lase | lase | other | | neuroprotecta |
| | | | | | | (HIF-PH) | (HIF-PH) | cardio- | | nt and cardio- |
| | | | | | | | Inhibitor | vascular | | protectant by |
| | | | | | | | | products | | binding to and |
| | | | | | | | | | | inhibits the |
| | | | | | | | | | | actions of |
| | | | | | | | | | | hypoxia- |
| | | | | | | | | | | inducible |
| | | | | | | | | | | factor (HIF) |
| | | | | | | | | | | prolyl |
| | | | | | | | | | | hydroxylase |
| | | | | | | | | | | (PH) and |
| | | | | | | | | | | protects |
| | | | | | | | | | | against tissue |
| | | | | | | | | | | damage or |
| | | | | | | | | | | injury. |
| | | | | | | | | | | FG4539 is |
| | | | | | | | | | | indicated for |
| | | | | | | | | | | the treatment |
| | | | | | | | | | | of acute |
| | | | | | | | | | | kidney failure, |
| | | | | | | | | | | ischemic |
| | | | | | | | | | | stroke and |
| | | | | | | | | | | myocardial |
| | | | | | | | | | | infarction. |
| FG4592 | ASP1517, | roxadustat | 808118- | 11256664 | Synthetic | Hypoxia- | Hypoxia- | other anti- | Isoquino- | FG4592 |
| | ASP1517, | | 40-3 | | | Inducible | Inducible | anaemic | line, | contains |
| | ASP1517, | | | | | Factor- | Factor- | products, | Isoquino- | roxadustat as |
| | AZD9941, | | | | | Prolyl | Prolyl | including | lines | an active |
| | AZD9941, | | | | | Hydroxy- | Hydroxy- | folic acid, | | ingredient. It |
| | FG4592, | | | | | lase | lase | folinic acid | | is an active |
| | FG4592053 | | | | | (HIF-PH) | (HIF-PH) | | | second |
| | | | | | | | Inhibitor | | | generation |
| | | | | | | | | | | hypoxia- |
| | | | | | | | | | | inducible |
| | | | | | | | | | | factor prolyl |
| | | | | | | | | | | hydroxylase |
| | | | | | | | | | | inhibitor that |
| | | | | | | | | | | activates HIF- |
| | | | | | | | | | | mediated |
| | | | | | | | | | | erythropoiesis. |
| | | | | | | | | | | FG4592 is |
| | | | | | | | | | | indicated for |
| | | | | | | | | | | the treatment |
| | | | | | | | | | | of anemia |
| | | | | | | | | | | associated |
| | | | | | | | | | | with chronic |
| | | | | | | | | | | renal failure. |
| FG6513 | FG6513 | — | — | — | — | Hypoxia- | Hypoxia- | other | — | FG-6513 is a |
| | | | | | | Inducible | Inducible | cardio- | | hypoxia- |
| | | | | | | Factor- | Factor- | vascular | | inducible |
| | | | | | | Prolyl | Prolyl | products | | factor prolyl |
| | | | | | | Hydroxy- | Hydroxy- | | | hydroxylase |
| | | | | | | lase | lase | | | inhibitor that |
| | | | | | | (HIF-PH) | (HIF-PH) | | | enhances |
| | | | | | | | Inhibitor | | | mobilization |
| | | | | | | | | | | of endothelial |
| | | | | | | | | | | progenitor |
| | | | | | | | | | | cells from the |
| | | | | | | | | | | bone marrow. |
| | | | | | | | | | | FG-6513 is |
| | | | | | | | | | | indicated for |
| | | | | | | | | | | the treatment |
| | | | | | | | | | | of ischemic |
| | | | | | | | | | | damage. |
| FG6515 | FG6515 | — | — | — | — | Hypoxia- | Hypoxia- | ALL | — | FG-6515 is a |
| | | | | | | Inducible | Inducible | OTHER | | hypoxia- |
| | | | | | | Factor- | Factor- | CNS | | inducible |
| | | | | | | Prolyl | Prolyl | DRUGS | | factor prolyl |
| | | | | | | Hydroxy- | Hydroxy- | | | hydroxylase |
| | | | | | | lase | lase | | | inhibitor |
| | | | | | | (HIF-PH) | (HIF-PH) | | | which induces |
| | | | | | | | Inhibitor | | | circulation of |
| | | | | | | | | | | endogenous |
| | | | | | | | | | | erythropoietin. |
| | | | | | | | | | | FG-6515 is |
| | | | | | | | | | | indicated for |
| | | | | | | | | | | protection |
| | | | | | | | | | | against |
| | | | | | | | | | | cerebral |
| | | | | | | | | | | ischemic |
| | | | | | | | | | | stroke. |
| FG6874 | — | — | — | — | — | Hypoxia- | Hypoxia- | anti- | — | FG6874 is a |
| | | | | | | Inducible | Inducible | anaemic | | novel HIF-PH |
| | | | | | | Factor- | Factor- | preparations | | (Hypoxia- |
| | | | | | | Prolyl | Prolyl | | | Inducible |
| | | | | | | Hydroxy- | Hydroxy- | | | Factor-Prolyl |
| | | | | | | lase | lase | | | Hydroxylase |
| | | | | | | (HIF-PH) | (HIF-PH) | | | (HIF-PH) |
| | | | | | | | Inhibitor | | | Inhibitor) |
| | | | | | | | | | | inhibitor. |
| | | | | | | | | | | FG6874 is |
| | | | | | | | | | | indicated for |
| | | | | | | | | | | the treatment |
| | | | | | | | | | | of anemia. |
| FG6888 | — | — | — | — | — | Hypoxia- | Hypoxia- | All other | — | FG6888 is a |
| | | | | | | Inducible | Inducible | antineo- | | HIF-PH |
| | | | | | | Factor- | Factor- | plastic- | | (Hypoxia- |
| | | | | | | Prolyl | Prolyl | cs | | Inducible |
| | | | | | | Hydroxy- | Hydroxy- | | | Factor-Prolyl |
| | | | | | | lase | lase | | | Hydroxylase |
| | | | | | | (HIF-PH) | (HIF-PH) | | | (HIF-PH) |
| | | | | | | | Inhibitor | | | Inhibitor) |
| | | | | | | | | | | inhibitor. |
| | | | | | | | | | | FG8205 |
| | | | | | | | | | | indicated for |
| | | | | | | | | | | the treatment |
| | | | | | | | | | | of cancer. |
| FG8205 | FG8205 | FG8205 | 122384- | 129710 | Synthetic | Hypoxia- | Hypoxia- | other | Benzodiaz- | FG8205 is a |
| | | | 14-9 | | | Inducible | Inducible | cardio- | epinones | HIF-PH |
| | | | | | | Factor- | Factor- | vascular | | (Hypoxia- |
| | | | | | | Prolyl | Prolyl | products | | Inducible |
| | | | | | | Hydroxy- | Hydroxy- | | | Factor-Prolyl |
| | | | | | | lase | lase | | | Hydroxylase |
| | | | | | | (HIF-PH) | (HIF-PH) | | | (HIF-PH) |
| | | | | | | | Inhibitor | | | Inhibitor) |
| | | | | | | | | | | selective |
| | | | | | | | | | | inhibitor. |
| | | | | | | | | | | FG8205 is |
| | | | | | | | | | | indicated for |
| | | | | | | | | | | the treatment |
| | | | | | | | | | | of |
| | | | | | | | | | | cardiovascular |
| | | | | | | | | | | failure. |
| GSK1278863 | GSK1278863, | daprodustat | 960539- | 91617630 | Synthetic | Hypoxia- | Hypoxia- | all other | Barbiturates | GSK1278863 |
| | GSK1278863A, | | 70-2 | | | Inducible | Inducible | therapeutic | | contains |
| | GSK1278863A | | | | | Factor- | Factor- | products, | | daprodustat as |
| | | | | | | Prolyl | Prolyl | anti- | | an active |
| | | | | | | Hydroxy- | Hydroxy- | anaemic | | ingredient and |
| | | | | | | lase | lase | preparations, | | is a novel |
| | | | | | | (HIF-PH) | (HIF-PH) | other | | small |
| | | | | | | | Inhibitor | cardio- | | molecule, |
| | | | | | | | | vascular | | hypoxia- |
| | | | | | | | | products | | inducible |
| | | | | | | | | wound | | factor-prolyl |
| | | | | | | | | healing | | hydroxylase |
| | | | | | | | | agents | | inhibitor, |
| | | | | | | | | | | which |
| | | | | | | | | | | increases the |
| | | | | | | | | | | production of |
| | | | | | | | | | | erythropoietin |
| | | | | | | | | | | and stimulates |
| | | | | | | | | | | erythropoiesis. |
| | | | | | | | | | | GSK1278863 |
| | | | | | | | | | | is indicated |
| | | | | | | | | | | for the |
| | | | | | | | | | | treatment of |
| | | | | | | | | | | anemia |
| | | | | | | | | | | associated |
| | | | | | | | | | | with chronic |
| | | | | | | | | | | renal failure, |
| | | | | | | | | | | peripheral |
| | | | | | | | | | | vascular |
| | | | | | | | | | | diseases, |
| | | | | | | | | | | ischemia, |
| | | | | | | | | | | diabetic foot |
| | | | | | | | | | | ulcer and |
| | | | | | | | | | | muscle |
| | | | | | | | | | | atrophy. |
| HIF- | HIFPHI | — | — | — | — | Hypoxia- | Hypoxia- | intestinal | — | HIF-Prolyl |
| Prolyl | | | | | | Inducible | Inducible | anti- | | Hydroxylase |
| Hydroxylase | | | | | | Factor- | Factor- | inflamm- | | Inhibitors |
| Inhibitor | | | | | | Prolyl | Prolyl | atory | | reduces the |
| FIBROGEN | | | | | | Hydroxy- | Hydroxy- | agents | | production of |
| | | | | | | lase | lase | | | inflammatory |
| | | | | | | (HIF-PH) | (HIF-PH) | | | mediators, |
| | | | | | | | Inhibitor | | | such as |
| | | | | | | | | | | interleukin |
| | | | | | | | | | | (IL)-6 and |
| | | | | | | | | | | monocyte |
| | | | | | | | | | | chemoattractant |
| | | | | | | | | | | protein |
| | | | | | | | | | | (MCP)-1, 10 |
| | | | | | | | | | | and inhibiting |
| | | | | | | | | | | the |
| | | | | | | | | | | differentiation |
| | | | | | | | | | | of pro- |
| | | | | | | | | | | inflammatory |
| | | | | | | | | | | Type 1 Helper |
| | | | | | | | | | | T Cells (THI) |
| | | | | | | | | | | cells from |
| | | | | | | | | | | naive |
| | | | | | | | | | | precursor T |
| | | | | | | | | | | cells. HIF- |
| | | | | | | | | | | prolyl |
| | | | | | | | | | | hydroxylase |
| | | | | | | | | | | Inhibitor is |
| | | | | | | | | | | indicated for |
| | | | | | | | | | | the treatment |
| | | | | | | | | | | of |
| | | | | | | | | | | inflammation |
| | | | | | | | | | | in chronic |
| | | | | | | | | | | neurodegenera- |
| | | | | | | | | | | tive conditions |
| | | | | | | | | | | such as |
| | | | | | | | | | | multiple |
| | | | | | | | | | | sclerosis, and |
| | | | | | | | | | | chronic |
| | | | | | | | | | | inflammatory |
| | | | | | | | | | | diseases, such |
| | | | | | | | | | | as |
| | | | | | | | | | | inflammatory |
| | | | | | | | | | | bowel disease. |
| JTZ951 | JTZ951 | — | — | — | — | Hypoxia- | Hypoxia- | anti- | — | JTZ951 is a |
| | | | | | | Inducible | Inducible | anaemic | | hypoxia |
| | | | | | | Factor- | Factor- | preparations | | inducible |
| | | | | | | Prolyl | Prolyl | | | factor-prolyl |
| | | | | | | Hydroxy- | Hydroxy- | | | hydroxylase |
| | | | | | | lase | lase | | | domain |
| | | | | | | (HIF-PH) | (HIF-PH) | | | inhibitor |
| | | | | | | | Inhibitor | | | which acts by |
| | | | | | | | | | | increasing red |
| | | | | | | | | | | blood cells by |
| | | | | | | | | | | accelerating |
| | | | | | | | | | | production of |
| | | | | | | | | | | erythropoietin, |
| | | | | | | | | | | an |
| | | | | | | | | | | erythropoiesis- |
| | | | | | | | | | | stimulating |
| | | | | | | | | | | hormone, via |
| | | | | | | | | | | inhibition of |
| | | | | | | | | | | HIF-PHD. |
| | | | | | | | | | | JTZ951 is |
| | | | | | | | | | | indicated for |
| | | | | | | | | | | the treatment |
| | | | | | | | | | | of anemia |
| | | | | | | | | | | associated |
| | | | | | | | | | | with chronic |
| | | | | | | | | | | kidney |
| | | | | | | | | | | disease. |
| PN3602 | HIFPH | — | — | — | — | Hypoxia- | Hypoxia- | haematinics, | — | PN3602 is a |
| | Modulator, | | | | | Inducible | Inducible | iron and all | | hypoxia- |
| | HIF | | | | | Factor- | Factor- | combin- | | inducible |
| | Stabilizer, | | | | | Prolyl | Prolyl | ations | | factor-prolyl |
| | PN3602 | | | | | Hydroxy- | Hydroxy- | | | hydroxylase |
| | | | | | | lase | lase | | | inhibitor |
| | | | | | | (HIF-PH) | (HIF-PH) | | | designed to |
| | | | | | | | Inhibitor | | | increase the |
| | | | | | | | | | | natural |
| | | | | | | | | | | production of |
| | | | | | | | | | | erythropoietin |
| | | | | | | | | | | and cause a |
| | | | | | | | | | | controlled, |
| | | | | | | | | | | gradual rise in |
| | | | | | | | | | | hemoglobin in |
| | | | | | | | | | | anemic |
| | | | | | | | | | | patients. |
| | | | | | | | | | | PN3602 is |
| | | | | | | | | | | indicated for |
| | | | | | | | | | | the treatment |
| | | | | | | | | | | of anemia. |
| ZYAN1 | ZYAN1 | — | — | — | — | Hypoxia- | Hypoxia- | ANTI- | — | ZYANI is a |
| | | | | | | Inducible | Inducible | ANAEMIC | | new molecular |
| | | | | | | Factor- | Factor- | PREPARA- | | entity and a |
| | | | | | | Prolyl | Prolyl | TIONS | | hypoxia- |
| | | | | | | Hydroxy- | Hydroxy- | | | inducible |
| | | | | | | lase | lase | | | factor-prolyl |
| | | | | | | (HIF-PH) | (HIF-PH) | | | hydroxylase |
| | | | | | | | Inhibitor | | | (HIF-PH) |
| | | | | | | | | | | inhibitor |
| | | | | | | | | | | which |
| | | | | | | | | | | increases the |
| | | | | | | | | | | production of |
| | | | | | | | | | | erythropoietin |
| | | | | | | | | | | and stimulates |
| | | | | | | | | | | erythropoiesis. |
| | | | | | | | | | | ZYANI is |
| | | | | | | | | | | indicated for |
| | | | | | | | | | | the treatment |
| | | | | | | | | | | of anemia. |

1. A method of increasing survival of a neuronal or photoreceptor cell in a subject in need thereof, the method comprising inhibiting or decreasing level and/or activity of prolyl hydroxylase (PHD) in the neuronal or photoreceptor cell.

2. The method of claim 1, wherein the neuronal or photoreceptor cell is a cone cell or a rod cell, or a combination of cone cells, rod cells, and/or other retinal cells.

3. The method of claim 1, wherein the inhibiting or decreasing comprises administering an effective amount of an inhibitor of PHD.

4. The method of claim 1, wherein the inhibitor of PHD is selected from the group consisting of proteins, nucleic acids, chemicals and combinations thereof.

5. The method of claim 3, wherein the inhibitor of PHD is Roxadustat (FG-4592), Vadadustat (AKB-6548), Daprodustat (GSK1278863), Desidustat (ZYAN-1), Molidustat (Bay 85-3934), MK-8617, YC-1, IOX-2, 2-methoxyestradiol, GN-44028, AKB-4924, Bay 87-2243, FG-2216, FG-4497, or combinations thereof.

6. The method of claim 3, wherein the inhibitor of PHD is a compound in Table 1.

7. The method of claim 4, wherein the nucleic acid is selected from the group consisting of an antisense oligonucleotide, a small interfering RNA (siRNA), a short hairpin RNA (shRNA), a guide RNA (gRNA) and combinations thereof.

8. The method of claim 1, wherein the inhibiting or decreasing comprises administering an effective amount of any combination of inhibitors of PHD.

9. The method of claim 1, wherein inhibiting or decreasing the level and/or activity of PHD further comprises altering glycolysis.

10-16. (canceled)

17. The method of claim 27, wherein the retinal degenerative disease is retinitis pigmentosa (RP), age-related macular degeneration (AMD), and/or glaucoma.

18. (canceled)

19. The method of claim 27, wherein the neurodegenerative disease is Alzheimer's disease, Parkinson's disease, Huntington's disease, Amyotrophic lateral sclerosis (ALS), and/or Lewy body dementia.

20-26. (canceled)

27. A method for treating a neurodegenerative or retinal degenerative condition in a subject, comprising administering a therapeutically effective amount of an inhibitor of prolyl hydroxylase (PHD) to the subject.

28. The method of claim 27, wherein the inhibitor of PHD is selected from the group consisting of proteins, nucleic acids, chemicals and combinations thereof.

29. The method of claim 27, wherein the inhibitor of PHD is Roxadustat (FG-4592), Vadadustat (AKB-6548), Daprodustat (GSK1278863), Desidustat (ZYAN-1), Molidustat (Bay 85-3934), MK-8617, YC-1, 10X-2, 2-methoxyestradiol, GN-44028, AKB-4924, Bay 87-2243, FG-2216, FG-4497, or combinations thereof.

30. The method of claim 27, wherein the inhibitor of PHD is a compound in Table 1.

31. The method of claim 28, wherein the nucleic acid is selected from the group consisting of antisense oligonucleotide, a small interfering RNA (siRNA), a short hairpin RNA (shRNA), a guide RNA (gRNA) and combinations thereof.

32. The method of claim 1, wherein the inhibitor of PHD is administered by intravitreal injection or subretinal injection.

33. (canceled)
